(19) United States
(12) Patent Application Publication
(10) Pub. No.: US 2015/0209518 A1
(43) Pub. Date: Jul. 30, 2015

(54) INJECTION DEVICE FOR METERING AND DISCHARGING A FIXED DOSE OF A LIQUID PRODUCT

(71) Applicant: TecPharma Licensing AG, Burgdorf (CH)

(72) Inventors: Ulrich Moser, Heimiswil (CH); Christian Schrul, Lyssach (CH); Markus Tschirren, Burgdorf (CH); Leos Urbanek, Bern (CH)

(21) Appl. No.: 14/681,021

(22) Filed: Apr. 7, 2015

Related U.S. Application Data
(63) Continuation of application No. PCT/CH2013/000176, filed on Oct. 8, 2013.

(30) Foreign Application Priority Data
Oct. 8, 2012 (CH) 01874/12

(51) Int. Cl. A61M 5/24; A61M 5/315

(52) U.S. Cl. CPC A61M 5/2425; A61M 5/31535

(57) ABSTRACT

An injection device for metering and discharging a fixed dose of a liquid product includes a metering element or a setting button for setting and for discharging a fixed dose by means of a guide element, and a holding element having a counter guide element and a first holding member and a second holding member. In an initial position, in a retracted and in a discharged position of the metering element or the setting button, the guide element of the metering element or of the setting button engages in a guiding manner with or engages in a slotted guide system with the counter guide element of the holding element.

### CROSS-REFERENCE TO RELATED APPLICATIONS

This application is a Continuation of International Patent Application No. PCT/CH2013/000176 filed Oct. 8, 2013, which claims priority to Swiss Patent Application No. 01874/12 filed Oct. 8, 2012, the entire contents of each are incorporated herein by reference.

### BACKGROUND

The invention relates to an injection device for metering and discharging a fixed dose of a liquid product.

Devices with which a fixed dose of liquid product can be metered and discharged are known from the prior art.

A fixed dose is normally understood to mean that the user cannot select the dose freely, but that the dose is predetermined and fixed.

WO2009/080775A1 describes an injection device comprising a first sleeve-like element and a second sleeve-like element, wherein the two elements are coupled to one another such that the metering movement and the discharging movement are performed in two steps. The first and second elements are brought into a guiding engagement during the metering movement and the discharging movement. During the discharging movement, a threaded rod is screwed into the injection device due to a rotationally fixed connection between the threaded rod and the second element.

Hereinafter, the distal position for an injection device for metering and discharging a fixed dose of a liquid product refers to the initial position of the injection device, in which the injection device is delivered, wherein a metering element or a setting button for setting and discharging a fixed dose is in its initial position, and the proximal position means the position in which the metering element or the setting button is in its cocked and injection-ready position. More generally, distal means a direction toward the needle end of the injection device or farther past it, and proximal means a position toward the setting button or farther past it.

### SUMMARY

A problem addressed by the invention is that of providing an injection device for metering and for discharging a fixed dose of a liquid product.

This problem is solved by the subject matter and advantageous embodiments of the claims.

The invention relates to an injection device for metering and for discharging a fixed dose of a liquid product, which has a metering element or a setting button for setting and discharging the fixed dose. For setting or metering the fixed dose, the metering element or the setting button can moved from a distal position, i.e., an initial position of the metering element or the setting button, into a proximal position, i.e., a cocked position of the metering element or the setting button, and to discharge the set dose, the metering element or the setting button can be moved back into a distal position, i.e., a discharged position of the metering element or the setting button. The metering element or the setting button has a guide element. The injection device further comprises a retaining element having a counter-guide element, wherein the guide element of the metering element or the setting button can be in a guiding engagement with the counter-guide element provided on a holding element or can be engaged with a slotted guide. The guiding engagement or the slotted guide can be designed such that the metering element or the setting button can be moved in the proximal direction during setting or metering of the fixed dose. During the metering movement or the retraction movement, which can be a rotational and/or axial movement in which the metering element or the setting button is moved in the proximal direction, and during the discharge movement, which can be a rotational and/or axial movement in which the metering element or the setting button is moved in the distal direction, the guide element can preferably be in the guiding engagement with the counter-guide element or in the slotted guide. In the initial position, in the retracted position and in the discharged position, the guide element of the metering element or the setting button can thus be in the guiding engagement with the counter-guide element with the retaining element or in the slotted guide.

The injection device further comprises a discharge element for discharging the fixed dose, with a first and a second recess. The retaining element further comprises a first and a second retaining member, wherein the first recess of the discharge element is suitable for receiving the first retaining member of the retaining element, or at least a part of the first retaining member of the retaining element, and the second recess of the discharge element is suitable for receiving the second retaining member, or at least a part of the second retaining member of the retaining element. These cooperating elements are preferably arranged pairwise or multiply, more particularly symmetrically.

The first recess of the discharge element cooperates with the first retaining member, or with at least a part of the first retaining member of the retaining element, and the second recess of the discharge element cooperates with the second retaining member, or with at least a part of the second retaining member of the retaining element in such a manner that, in an initial position of the metering element or the setting button, in which the metering element or the setting button is in a distal position, the first retaining member of the retaining element or at least a part of the first retaining member of the retaining element is engaged with the first recess of the discharge element, and in a retracted position of the metering element or the setting button, in which the metering element or the setting button is in a distal position, the second retaining member or at least a part of the second retaining member of the retaining element is engaged with the second recess of the discharge element.

This arrangement of the injection device is used for metering and discharging the fixed dose of a liquid product from the injection device. Particularly preferably, the arrangement of the injection device can be aligned such that multiple, in particular equal, fixed doses of the liquid product can be discharged.

The injection device can preferably comprise a metering element and a setting button, wherein the metering element and the setting button are coupled indirectly or directly to one another.

The metering element preferably further comprises a cutout, which is formed such that, in the discharge position of the metering element or the setting button, the first retaining member or at least a part of the first retaining member of the retaining element is engaged with the cutout of the metering element. In addition, the first retaining member or at least a part of the first retaining member of the retaining element can be formed such that it is disengaged from the first recess of the discharge element in the discharged position of the metering element or the setting button.

Alternatively, the metering element can comprise multiple cutouts. The cutouts can be arranged in the metering element offset from one another in the circumferential direction and preferably in the axial direction. The cutouts are advantageously arranged pairwise or multiply, in particular symmetrically. The axial distance between successive cutouts can determine the fixed dose.

In addition, the metering element can preferably be in sliding contact with the first retaining member or at least a part of the first retaining member in the initial position of the metering element or the setting button. It can be ensured by means of this contact that the first retaining member or at least a part of the first retaining member of the retaining element can be engaged with the first recess of the discharge element in the initial position of the metering element or the setting button. The sliding surface of the metering element can act upon the first retaining member or on at least a part of the first retaining member in such a manner that the first retaining member or at least a part of the first retaining member of the retaining element is elastically prestressed or deflected radially inward, wherein the first retaining member, or at least a part of the first retaining member, of the retaining element can be kept engaged with the first recess of the discharge element. Alternatively, the sliding surface of the metering element can act upon the first retaining member, or upon at least a part of the first retaining member, in such a manner that the first retaining member, or at least a part of the first retaining member, of the retaining element is prevented from being elastically prestressed or deflected radially outward, so that the first retaining member or at least a part of the first retaining member of the retaining element can remain engaged with the first recess of the discharge element. As described, the individual elements participating in this interaction can advantageously each be arranged multiply, more particularly symmetrically, for example, facing one another pairwise.

In addition, the metering element can be in sliding contact with the first retaining member or with at least a part of the first retaining member in the retracted position of the metering element or the setting button. Analogously to the above-mentioned initial position of the metering element or the setting button, the sliding surface of the metering element can act on the first retaining member or on at least a part of the first retaining member in such a manner that the first retaining member or at least a part of the first retaining member of the retaining element is elastically prestressed or deflected radially inward or is prevented from being elastically prestressed or deflected radially outward. Therefore it is ensured that, during the dose-metering process for setting the fixed dose, the first retaining member or at least a part of the first retaining member of the retaining element can remain engaged with the discharge element, so that at least no relative axial movement between the first retaining element and the discharge element can take place.

The metering element or the setting button can be rotatable relative to the retaining element. The guide element of the metering element or the setting button is engaged with the counter-guide element of the retaining element, particularly in a guiding engagement or in a slotted guide, wherein the metering element or the setting button is rotatable relative to the retaining element. Particularly preferably, the metering element or the setting button can be moved by this rotational movement in the proximal direction relative to the retaining element, so that a rotational and axial relative movement between the metering element or the setting button and the retaining element can be carried out.

The injection device can further comprise a drive means, which is designed such that it can act on the discharge element. It is particular preferred if the drive means, particularly a spring, is prestressed in the initial position of the metering element or the setting button. That is to say, the user need not cock the injection device, but instead receives a device with an already prestressed drive means, more particularly a spring.

The discharge element can be sleeve-shaped. In addition, the prestressed drive means can be received at least in part by the sleeve-like discharge element. Thereby the construction of the injection device can be reduced in size and/or the drive means can be guided and/or stabilized.

The first and second recesses of the discharge element are arranged relative to the first and second retaining member or at least a part of the first or the second retaining member of the retaining element in such a manner that the fixed dose can be discharged from the device. The relative arrangement is designed in particular such that a plurality of identically fixed doses can be discharged from the injection device.

The first and second recesses of the discharge element are particularly preferably arranged offset relative to one another in the circumferential direction and preferably in the axial direction. The cutouts of the first and second recesses are preferably arranged at an equal axial height in such a manner that they overlap in the circumferential direction. This overlapping of the two recesses can be designed such that both the first and also the second retaining member or at least a part of the first or the second retaining member of the retaining element can be engaged, in at least one position of the metering element or the setting button relative to the retaining element, in the corresponding recesses of the discharge element. As described, the individual elements participating in this interaction can advantageously each be arranged multiply, more particularly symmetrically, for example, facing one another pairwise.

Alternatively, the first and the second recesses of the discharge element can be provided offset from one another in the circumferential direction and at the same height in the axial direction.

Alternatively, the first and the second retaining member or at least a part of the first or the second retaining member of the retaining element, or the cutout of the metering element or the multiple cutouts of the metering element can be designed such that, in at least one position of the metering element or the setting button relative to the corresponding cutout of the discharge element, both the first and second retaining member or at least a part of the first or the second retaining member of the retaining element are engaged with the corresponding cutout of the discharge element. As described, the individual elements participating in this interaction can advantageously each be arranged multiply, more particularly symmetrically, for example, facing one another pairwise.

The retaining element can be sleeve-like in shape. The retaining element can, at least partially, receive the discharge element with the drive means, in particular the spring, arranged therein.

The first and second retaining members or at least a part of the first and second retaining members of the retaining element can be arranged at the same axial height and offset from one another in the circumferential direction. Alternatively, the first and second retaining members or at least a part of the first or second retaining members of the retaining element can be arranged offset from one another in the circumferential direction and in the axial direction. In addition, the retaining elements, as well as the retaining members or at least a part of the retaining members, can be formed elastically. Especially preferably, the retaining elements and the retaining members or at least a part of the retaining members can be movable radially inward and/or radially outward, and in particular can be tensioned or deflected radially inward or radially outward. The retaining elements, as well as the retaining members or at least a part of the retaining members, are designed such that they can engage with the corresponding recess of the discharge element and prevent an axial displacement of the discharge element, particularly in the distal direction.

The retaining elements, as well as the retaining members or at least a part of the retaining members, can be formed as a snap arm or snap tongue. The snap arms or snap tongues are designed such that they can simultaneously or alternately engage with the corresponding recess of the discharge element such that an axial displacement can be enabled, particularly in the distal direction of the discharge element. In particular, the snap arms or the snap tongues can comprise a snap hook or a snap cam or another protrusion, which can engage with the corresponding recesses of the discharge element.

In addition, the guide element of the metering element or of the setting button can be formed as a guide cam, and the counter-guide element of the retaining element as a slotted guide. Alternatively, the guide element of the metering element or of the setting button can be formed as a slotted guide, and the counter-guide element of the retaining element as a guide cam.

The guide cam and the slotted guide have an engagement or a guiding engagement in such a manner that, in the initial position, the retracted position and the discharge position of the metering element or the setting button, the cutout of the metering element is configured in relation to the first and second retaining member or to a part of the first and second retaining member of the retaining element in such a manner that the fixed dose can be discharged. For this purpose, the cutout of the metering element can interact in the corresponding position of the metering element with one of the retaining members, or with at least a part of the retaining members of the retaining element, in such a manner that the one retaining member or at least a part of the retaining member of the retaining element can protrude at least partially into the cutout or into the at least one cutout and can elastically relax or tension itself or can radially deflect elastically into the cutout or into the at least one cutout, while the other retaining member or at least a part of the other retaining member of the retaining element is prevented from protruding into the cutout or into the at least one cutout. Alternatively, the other retaining member or at least a part of the other retaining member of the retaining element can protrude into the cutout, analogously to the first retaining member or to at least a part of the first retaining member.

The injection device can further comprise a housing, which is indirectly or directly connected axially and rotationally fixedly to the retaining element. The sleeve-like housing can receive the sleeve-like retaining element at least in part, wherein the sleeve-like metering element can be arranged between the housing and the retaining element. The metering element can be rotationally and preferably axially movable relative to the housing and the retaining element. By a rotational and preferably axial movement of the metering element relative to the retaining element, the first and/or the second retaining members, or at least a part of the first and/or the second retaining members, can move into the cutout or into the corresponding cutout of the metering element such that the first and/or the second retaining element, or at least a part of the first and/or second retaining element, correspondingly disengage from the first and/or second cutout of the discharge element, and the discharge element can be moved in the distal direction in order to output the fixed dose from the injection device. Alternatively, the corresponding fixed dose can be output only by a rotational movement of the metering element relative to the retaining element.

Alternatively, the metering element can be arranged indirectly or directly axially fixedly relative to the housing and/or the retaining element. Additionally, the metering element can be rotatable only in one direction relative to the housing and/or the retaining element due to a reverse-rotation lock mechanism. By a rotational movement of the metering element relative to the retaining element, the first and/or the second retaining members or at least a part of the first and/or second remaining members of the retaining element can consequently move into the cutout of the metering element such that the first and/or second retaining members or at least a part of the first and/or second retaining members of the retaining element correspondingly disengage from the first and/or second cutout of the discharge element. This can have the result that the discharge element can be moved in the distal direction in order to output the fixed dose from the injection device.

The injection device further comprises a carpule, in which the liquid product to be dispensed is located. The carpule can additionally be received by a carpule holder. The carpule or the carpule holder can be axially and rotationally fixedly connected to the housing or the retaining element indirectly or directly. The carpule or the carpule holder is preferably positioned at the distal end of the housing or the distal end of the retaining element. An injection needle for discharging the liquid product can be mounted on the carpule or the carpule holder via a needle connection, particularly a rotary connection, a bayonet connection or a snap connection in the form of a connecting element, and a mating connecting element can be mounted on the injection needle. A stopper is displaceably received in the carpule so that the liquid product can be discharged from the carpule through the injection needle. The discharge element can act on the stopper such that the stopper can be moved relative to a wall of the carpule. The discharge element can act on the stopper directly or indirectly, particularly via a displacement element.

The injection device can comprise a setting button. This setting button can be used for setting and/or discharging the fixed dose on the injection device. This setting button can be coupled indirectly or directly to the metering element such that the metering element can be controlled by actuating the setting button. In addition, the injection device can have a mechanism with which a dose correction, particularly a reverse rotation contrary to a metering direction, can be prevented.

The setting button can be axially fixedly and rotationally fixedly connected to the metering button, indirectly or directly, and surround a housing of the injection device at least in part, so that the user can grip the setting button. The setting button can also comprise a gripping device, which is used so that the setting button can be gripped better. The rotational movement of the setting button can be transferred to the metering element by a rotation of the setting button, relative to the housing, by the user. During the setting or metering of the fixed dose, the metering element and the setting button can be moved in the proximal direction relative to the housing, due to a guiding engagement or a slotted guide between the metering element and the retaining element fixed relative to the housing, and due to the indirect or direct axially fixed connection between the setting button and the metering element.

Alternatively, the setting button can be connected to the housing, indirectly or directly, axially fixedly and rotatably relative to the housing. The setting button can be rotationally fixedly connected to the metering element indirectly or directly, the metering element being axially movable relative to the setting button. Thus the rotational movement of the setting button can likewise be transmitted to the metering element by rotation of the setting button by the user. Therefore, the metering element can move in the proximal direction in a relative rotation movement between the setting button and the housing during the setting or metering of the fixed dose and due to a guiding engagement and a slotted guide between the metering element and the retaining element.

Alternatively, the setting button can be indirectly or directly connected to the metering element rotationally fixedly, the setting button being movable axially relative to the metering element. The setting button can additionally be connected so as to be rotatable relative to the housing. Consequently, the setting button can be moved relative to the housing in the proximal direction by means of a relative rotational movement of the setting button by the user during the setting or metering of the fixed dose and due to a guiding engagement or a slotted guide between the setting button and the retaining element.

The injection device can further comprise a display device, which can display the initial position, the retracted position and/or the discharge position of the metering element or the setting button. The display device can be designed as a visual, acoustic or tactile display. The visual display device can comprise a marking and/or a display digit and/or an intermediate space between two display digits.

For that purpose, the injection device can comprise a display sleeve with a display device. The display sleeve can be connected to the housing via a threaded connection and, indirectly or directly, to the metering element or the setting button via a rotationally fixed connection. Thus the display sleeve can undergo a rotational and axial movement relative to the housing during the metering movement or the rotational movement of the metering element or the setting button. The display sleeve can preferably be screwed in the distal direction during the retracting movement or the rotational movement of the metering element of the setting button. A reverse rotation lock mechanism can also be provided, which has the effect that the metering element or the setting button can be rotated only in one direction relative to the housing. For that purpose, a reverse rotation lock, or at least part of a reverse rotation lock, which can cooperate with a different part of the injection device, can be provided on the metering element or on the setting button or on the display sleeve.

Alternatively, a carpule or carpule sleeve can have a display device that can indicate the relative position of the stopper during the discharge of the liquid product. During discharging, a discharge element acts on a stopper in the carpule, whereby the stopper is axially movable relative to the wall of the carpule. Thus, the relative position of the stopper can be determined by an at least partially transparent carpule or carpule holder, wherein the display device on the carpule or the carpule sleeve, as well as the position of the stop, can additionally be displayed.

Alternatively, the display sleeve with the display device can have a threaded connection to the housing and a different threaded connection to a metering element. The two threaded connections are preferably formed in opposite directions and with different thread pitches. It would also be possible for the two threaded connections to be formed in the same direction and with different thread pitches. The display sleeve can be connected via an internal thread to an external thread of the metering element and via an external thread to an internal thread of the housing. The thread pitch of the threaded connection between the display sleeve and the housing can preferably be less steep than the thread pitch of the threaded connection between the display sleeve and the metering element. It is particularly preferred if the threaded connection between the display sleeve and the metering element is not self-locking and the threaded connection between the display sleeve and the housing is self-locking or not self-locking. By rotating the metering element during the metering movement, wherein the metering element can alternatively be rotated by the user via a setting button rotationally fixedly connected to the metering element, the display sleeve is axially displaced in the distal direction relative to the housing by the two threaded connections. The pitch of a counter-guide element, particularly the slotted guide of the retaining element, can preferably be smaller than the pitch of the threaded connection between the metering element and the display sleeve. Therefore, the display sleeve can be screwed in the distal direction relative to the housing when setting or metering the fixed dose, wherein a rotational and axial movement of the display sleeve relative to the housing can be performed. When discharging the fixed dose, the metering element, which can alternatively be axially fixedly connected to the setting button, can be displaced relative to the housing in the axial direction, more particularly in the distal direction. The display sleeve can thus be moved rotationally relative to the housing and axially in the distal direction due to the threaded connection and the axial driving of the metering element. Preferably, the display sleeve can have the same angle of rotation or undergo the same axial displacement during the setting or metering of the fixed dose as during the discharging of the fixed dose.

Alternatively, the display sleeve with the display device can have a threaded connection to the housing and a different threaded connection to the setting button. The display sleeve can have an external thread, which is in a threaded engagement with an internal thread of the housing, and an internal thread, which is in a threaded engagement with an external thread of the setting button. The two threaded connections are preferably formed in opposite directions and with different thread pitches. It would also be possible for the two threaded connections to be formed in the same direction and with different thread pitches. The thread pitch of the threaded connection between the display sleeve and the setting button is preferably steeper than the thread pitch of the threaded connection between the display sleeve and the housing. It is particularly preferred if the threaded connection between the display sleeve and the setting button is not self-locking and the threaded connection between the display sleeve and the housing is self-locking or not self-locking. An indirect or direct rotationally fixed connection can be provided between the display sleeve and the metering element. During the ejection movement, in which the setting button can be moved axially in the distal direction, the display sleeve can be moved rotationally in the housing in the axial direction due to the axial force acting on the display sleeve, wherein the metering element can also be rotated by the rotationally fixed connection. The pitch of a counter-guide element, particularly the slotted guide of the retaining element, can preferably be equally large as the pitch of the threaded connection between the display sleeve and the setting button.

The injection device can further comprise a mechanism that can display the end of a discharge of the fixed dose. The display device for indicating the end of discharging the fixed dose can be visual, acoustic or tactile. The visual display device can comprise a marking and/or a display digit and/or an intermediate space between two display digits.

For this purpose, the injection device can comprise an additional drive means, particularly a spring, preferably prestressed. This spring can be prestressed between the carpule and a proximal tensioning element, or between a distal and a proximal tensioning element. The distal tensioning element can be supported on the carpule. In order to prevent an axial play between the carpule and the carpule holder, the spring can act, directly or via the distal tensioning element, on the carpule in such a manner that the carpule can be pressed in the distal direction against a carpule holder that is connected axially and rotationally fixedly in relation to a housing. The proximal tensioning element, or alternatively the distal and proximal tensioning elements, can be mounted slidably on the discharge element. In the initial position of the metering element or the setting element, the proximal tensioning element can be supported with an end face of a stop against a stop of the retaining element, wherein a radially inward-protruding snapper that is mounted on the proximal tensioning element protrudes into a distal recess of the discharge element. During the process of discharging the fixed dose from the injection device, the discharge element moves in the distal direction, wherein an end face of the snapper on the proximal tensioning element can abut axially against an end face of a connecting web of two recesses of the discharge element that are arranged offset from one another. This stopping contact can have the effect that the proximal tensioning element can be moved in the distal direction, tensioning or further tensioning the spring. The proximal tensioning element can be moved in the distal direction until a groove provided on a retaining sleeve or a shoulder provided on a retaining sleeve comes into alignment with the snapper on the proximal tensioning element and the snapper can be deflected radially outwardly, the spring being kept pretensioned due to a stop between end face of the groove on the shoulder and an end face of the snapper on the proximal tensioning element. In the further process of discharging the fixed dose from the injection device, the connecting web of the discharge element, which is provided between two recesses of the discharge element arranged offset from one another in the axial direction, namely the distal and a proximal recess, slides along the snapper of the proximal tensioning element until the snapper of the proximal tensioning element snaps into the proximal recess. At the end of the process of discharging the fixed dose from the injection device, the spring between the carpule and the proximal tensioning element or between the distal and proximal tensioning elements relaxes, the proximal tensioning element then being able to strike with the end face of the stop against the end face of the stop on the retaining element due to the spring force of the spring acting on the proximal tensioning element. This stopping contact can trigger an acoustic signal, in particular a click sound.

Alternatively, an acoustic signal, particularly a click sound, can also be produced by the elastic radially inward deflection or relaxation of the snapper on the proximal tensioning element into the proximal recess. The snapper of the proximal tensioning element can strike against a connecting web of the discharge element provided between two recesses of the discharge element that are axially offset from one another, so that an acoustic signal, particularly a click, can be triggered.

An additional drive means, in particular a preferably pretensioned spring braced distally against a retaining element fixed relative to the housing and proximally against a proximal tensioning element, can alternatively be provided in order to indicate the end of discharging the fixed dose. The proximal tensioning element is non-rotatably connected to the retaining element, wherein the proximal tensioning element is arranged so as to be displaceable on the retaining element. The proximal tensioning element is prestressed by the spring force against a distal end face of the metering element. A proximal end face of the proximal tensioning element and the distal end face of the metering element are formed in sawtooth shapes that are complementary to one another and can interlock with one another. In the initial position of the metering element or the setting button, the two sawtooth-shaped stop surfaces of the proximal tensioning element and the metering element are engaged with one another. The metering element can rotate relative to the retaining element during the process of discharging the fixed dose from the injection device. By means of the sawtooth-like engagement between the proximal tensioning element and the metering element and the indirect or direct rotationally fixed connection between the proximal tensioning element and the retaining element, the proximal tensioning element can be moved in the distal direction so that the spring is tensioned or further tensioned. A retaining member of the retaining element moves due to the relative rotational movement of the metering element into a cutout of the metering element, wherein an axial movement of the proximal tensioning element in the proximal direction can be prevented by the stopping contact between a proximal end face of the proximal tensioning element and an end face of the retaining member. The retaining member of the retaining element is elastically prestressed or deflected radially outward into the cutout of the metering element by the axial pressure from the spring in the discharge element, which spring acts on the retaining member of the retaining element, via the connecting web of the discharge element, which connecting web acts on the retaining member of the retaining element between two mutually offset cutouts of the discharge element, namely a distal and a proximal cutout of the discharge element. In the further process of discharging the fixed dose from the injection device, the connecting web of the discharge element slides along the retaining member until the retaining member of the retaining element moves into the proximal recess and relaxes or elastically deflects. Due to the force of the prestressed spring, the proximal tensioning element can strike with its sawtooth-shaped stop surface against the sawtooth-shaped stop surface of the metering element. This stop can trigger an acoustic signal, in particular a click sound.

The injection device can additionally provide a mechanism that can prevent setting of a further dose if the final dose has been discharged.

For this purpose, a display sleeve of the injection device can comprise a stop that strikes against a stop provided in the housing. Therefore an axial and/or radial stopping contact between the display sleeve and the housing can prevent the setting or metering of an additional dose when the final dose has been discharged.

Alternatively, an axial and/or radial stopping contact between a housing and a metering element or a setting button can be provided, which prevents the setting or metering of an additional dose when the final dose has been discharged.

Alternatively, a retaining member, or at least a part of the retaining member, of the retaining element can protrude under elastic pretension into a recess of the metering element or can be elastically deflected radially outward into a recess, the discharge element having no further recess for the relaxation of the retaining member. The retaining member can be pressed by the discharge element radially into the recess of the metering element. The retaining member or at least a part of the retaining member of the retaining element comes into stopping contact with one side of the recess in the metering element, so that rotation of the metering element relative to the retaining element can be prevented. Thus the setting or metering of an additional dose can be prevented when the final dose has been discharged.

### BRIEF DESCRIPTION OF THE DRAWINGS

FIG. 1 shows an exploded view of a first embodiment of an injection device.

FIG. 2a shows a longitudinal sectional view of the first embodiment of the injection device in an initial position of a metering element (9).

FIG. 2b shows the injection device according to FIG. 2a rotated by 90° about the longitudinal axis B-B, in a longitudinal section.

FIG. 3a shows a longitudinal sectional view of the first embodiment of the injection device in a retracted position of the metering element (9).

FIG. 3b shows the injection device according to FIG. 3a rotated by 90° about the longitudinal axis B-B, in a longitudinal section.

FIG. 4 shows a detailed view of the proximal end of the injection device according to FIGS. 3a and 3b, wherein a part of the housing (10) and a part of the setting button (11) are cut away so that the interior of the injection device is visible.

FIG. 5a shows a longitudinal sectional view of the first embodiment of the injection device in an injection-triggered position of the metering element (9).

FIG. 5b shows the injection device according to FIG. 5a rotated by 90° about the longitudinal axis B-B, in a longitudinal section.

FIG. 6a shows a detailed view of the injection device according to FIG. 5b, wherein the mechanism for indicating the end of injection is shown in the injection-triggered position of the metering element (9).

FIG. 6b shows a detailed view of the injection device according to FIG. 6a, wherein the mechanism for indicating the end of injection is shown in the intermediate position between the injection-triggered position and the injection-ended position of the metering element (9).

FIG. 6c shows a detailed view of the injection device according to FIG. 6a, wherein the mechanism for indicating the end of injection is shown in the injection-ended position of the metering element (9).

FIG. 7a shows a longitudinal sectional view of the first embodiment of the injection device in a completely discharged position of the metering element (9).

FIG. 7b shows the injection device according to FIG. 7a rotated by 90° about the longitudinal axis B-B, in a longitudinal section.

FIG. 8 shows an exploded view of a second embodiment of an injection device.

FIG. 9a shows a longitudinal sectional view of the second embodiment of the injection device in an initial position of a metering element (90).

FIG. 9b shows the injection device according to FIG. 9a rotated by 90° about the longitudinal axis B-B, in a longitudinal section.

FIG. 10a shows a longitudinal sectional view of the second embodiment of the injection device in a retracted position of the metering element (90).

FIG. 10b shows the injection device according to FIG. 10a rotated by 90° about the longitudinal axis B-B, in a longitudinal section.

FIG. 10c shows a detailed view of the proximal end of the injection device according to FIGS. 10a and 10b, wherein a part of the housing (100) and a part of the setting button (110) are cut away so that the interior of the injection device is visible.

FIG. 11a shows a longitudinal sectional view of the second embodiment of the injection device in an injection-triggered position of the metering element (90).

FIG. 11b shows the injection device according to FIG. 11a rotated by 90° about the longitudinal axis B-B, in a longitudinal section.

FIG. 12 shows a detailed view of the proximal end of the injection device of the second embodiment in a completely discharged position of the metering element (90), wherein a part of the housing (100) and a part of the setting button (110) are cut away so that the interior of the injection device is visible.

FIG. 13 shows an exploded view of a third embodiment of an injection device.

FIG. 14a shows a longitudinal sectional view of the third embodiment of the injection device in an initial position of a setting button (1100).

FIG. 14b shows the injection device according to FIG. 14a rotated by 90° about the longitudinal axis B-B, in a longitudinal section.

FIG. 15a shows a longitudinal sectional view of the third embodiment of the injection device in a retracted position of the setting button (1100).

FIG. 15b shows the injection device according to FIG. 15a rotated by 90° about the longitudinal axis B-B, in a longitudinal section.

FIG. 16 shows a detailed view of the proximal end of the injection device according to FIGS. 15a and 15b, wherein a part of the housing (1000) and a part of the setting button (1100) are cut away so that the interior of the injection device is visible.

FIG. 17a shows a longitudinal sectional view of the third embodiment of the injection device in an injection-triggered position of the setting button (1100).

FIG. 17b shows the injection device according to FIG. 17a rotated by 90° about the longitudinal axis B-B, in a longitudinal section.

FIG. 18 shows a detailed view of the proximal end of the injection device of the second embodiment in a completely discharged position of the setting button (1100), wherein a part of the housing (1000) and a part of the setting button (1100) are cut away so that the interior of the injection device is visible.

FIG. 19a shows a detailed view of an injection device, wherein the mechanism for displaying the end of the injection is shown in an initial position of the setting button.

FIG. 19b shows a longitudinal sectional view of the injection device according to FIG. 19a.

FIG. 20a shows a detailed view of the injection device according to FIG. 19a, wherein the mechanism for indicating the end of injection is shown in the intermediate position between the injection-triggered position and the injection-ended position of the setting button.

FIG. 20b shows the longitudinal sectional view of the injection device according to FIG. 20a.

FIG. 21a shows a detailed view of the injection device according to FIG. 19a, wherein the mechanism for indicating the end of injection is shown in the injection-ended position of the setting button.

FIG. 21b shows the longitudinal view of the injection device according to FIG. 21a.

FIG. 22 shows an exploded view of a fourth embodiment of an injection device.

FIG. 23a shows a longitudinal sectional view of the fourth embodiment of the injection device in an initial position of a metering element (9000).

FIG. 23b shows the injection device according to FIG. 23a rotated by 90° about the longitudinal axis B-B, in a longitudinal section.

FIG. 24a shows a longitudinal sectional view of the fourth embodiment of the injection device in a retracted position of a metering element (9000).

FIG. 24b shows the injection device according to FIG. 24a rotated by 90° about the longitudinal axis B-B, in a longitudinal section.

FIG. 25a shows a longitudinal sectional view of the fourth embodiment of the injection device in an injection-triggered position of the metering element (9000).

FIG. 25b shows the injection device according to FIG. 25a rotated by 90° about the longitudinal axis B-B, in a longitudinal section.

### DETAILED DESCRIPTION

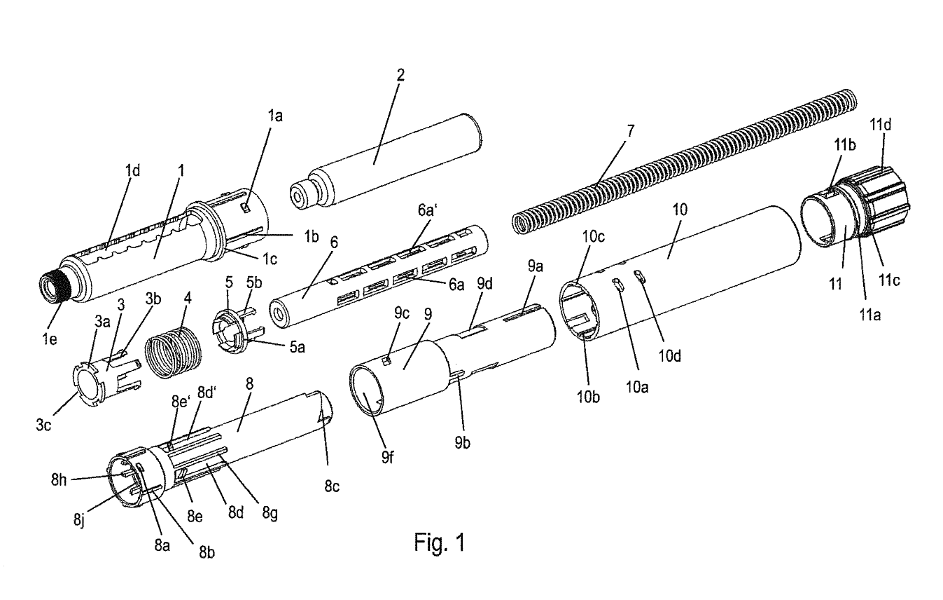

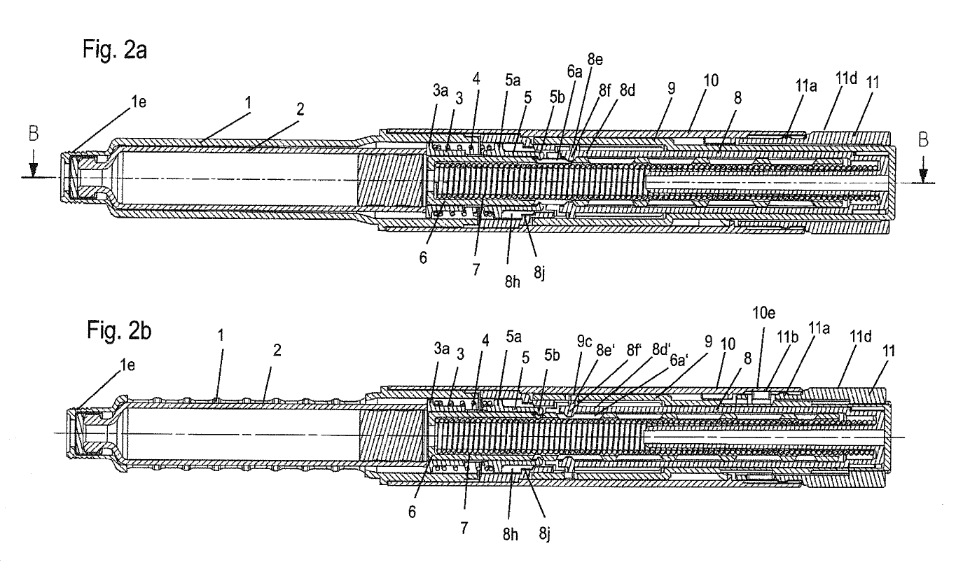

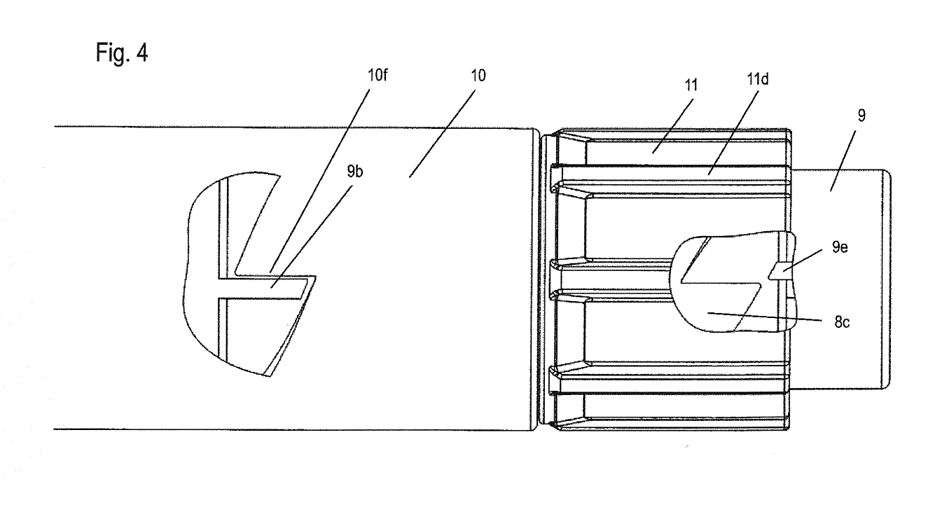

FIG. 1 shows an exploded view of a first embodiment of an injection device according to the invention. The injection device comprises a carpule (2) that is received by a carpule holder (1), wherein the carpule holder (1) is axially fixedly connected via a carpule holder tab (1a) to a distal housing cutout (10a) of a housing (10). A carpule holder web (1b) for rotationally fixed connection to the housing (10) via a corresponding housing groove (10b) is also provided on the carpule holder (1). A carpule holder stop (1c) can strike axially against a distal housing edge (10c) of the housing (10). A carpule holder cutout (1d) in the carpule holder (1) is used for displaying the relative position of an axially movable stopper in the carpule (2). A needle-connecting element (1e) that is provided for detachably affixing an injection needle is provided at the distal end of the carpule holder (1). The injection device further comprises a metering element (9), which is radially arranged between the housing (10) and a retaining element (8). The retaining element (8) is axially fixedly connected to a proximal housing tab cutout (10d) of the housing (10) with the aid of the housing element tab (8a). A housing element web (8b) for rotationally fixed connection to a corresponding distal housing groove (10b) is provided on the retaining element (8). The injection device additionally comprises a setting button (11), which protrudes at least in part into the interior of the sleeve-like housing (10) and is axially fixedly connected by a setting button ring (11a) mounted on the setting button (11) to the housing (10) via a ring/groove connection. On the setting button (11), a setting button snap arm (11b) is provided, which protrudes radially outward and can cooperate with a proximal housing groove or housing ramp (10e; visible in FIG. 2b, for example), provided on the inner circumferential surface of the housing (10), in such a manner that the setting button (11) is rotatable relative to the housing (10) only in one direction. The engagement of the setting button snap arm (11b) with the proximal housing groove or housing ramp (10e; visible in FIG. 2b, for example) can trigger an acoustic signal, more particularly a click sound. A setting button handle web (11d) for setting or metering the dose extends in the longitudinal direction on the outer circumferential surface of the setting button (11). The setting button snap arm (11b) can elastically deflect radially inward into a metering element depression (9d) provided on the outer circumferential surface of the metering element (9). The setting button (11) has, on the inner circumferential surface thereof, a setting button web (11c), which forms a rotationally fixed connection to a proximal metering element groove (9a) provided on the outer circumferential surface of the metering element (9). A metering element cam (9e; visible in FIG. 4) is provided on the inner circumferential surface of the metering element (9) and may serve as a guide element and, upon rotation of the metering element (9), can slide, via the rotationally fixed connection of the setting button (11), along a sawtooth-shaped retaining element control cam (8c) positioned on the outer circumferential surface of the retaining element (8). The metering element cam (9e; visible in FIG. 4) of the metering element (9) and the retaining element control cam (8c) of the retaining element (8) can cooperate as a guide element and a counter-guide element, respectively, such that the metering element (9) can be moved axially in the proximal direction upon rotation of the metering element (9) relative to the retaining element (8). On the outer circumferential surface of the metering element (9), a metering element web (9b) is provided, which can be in a guiding engagement or in a slotted guide with a sawtooth-shaped housing control cam (10f; visible in FIG. 4) that is positioned on the inner circumferential surface of the housing (10). The metering element web (9b) of the metering element (9) and the housing control cam (10f; visible in FIG. 4) of the housing (10) can cooperate in such a manner that more than one fixed dose to be administered can be set upon rotation of the metering element (9) relative to the housing (10). The metering element (9) has a metering element cutout (9c), which is designed such that a snap cam (8e; 8e′), which is formed extending radially outward on a retaining member (8d; 8d′) of the retaining element (8), can protrude into this metering element cutout (9c). The metering element (9) comprises two metering element cutouts (9c), which are offset relative to one another by approximately 180° in the circumferential direction of the metering element (9). The retaining member (8d; 8d′) of the retaining element (8) can be constructed as a snap arm. The retaining element (8) has a first retaining member (8d) and a second retaining member (8d′) or a first or second retaining element snap arm, wherein the first retaining member (8d) or the first snap arm comprises a first outward-protruding snap cam (8e) and a first inward-protruding snap cam (8f; visible in FIG. 2a), and the second retaining element member (8d′) or the second snap arm comprises a second outward-protruding snap cam (8d′) and a second inward-protruding snap cam (8f′; visible in FIG. 2b). The first outward-protruding snap cam (8e) and the second outward-protruding-snap cam (8e′) and the first inward-protruding snap cam (8f) and the second inward-protruding snap cam (8f′) are arranged at approximately equal axial height on the retaining element (8). The first inward-protruding snap cam (8f) is arranged on the first retaining member (8d) approximately diametrically opposite the first outward-protruding snap cam (8e), while the second inward-protruding snap cam (8f′) is provided on the second retaining member (8d′) approximately diametrically opposite the second outward-protruding snap cam (8e′). The first retaining member (8d) and the second retaining member (8d′) are arranged relative to one another at approximately a 90° angle in the circumferential direction of the retaining element (8). In addition, two first retaining members (8d) and two second retaining members (8d′) are provided on the retaining element (8), wherein the two first retaining members (8d) are arranged at approximately a 180° angle relative to one another in the circumferential direction of the retaining element (8) and the two second retaining members (8d′) are arranged at approximately a 180° angle relative to one another in the circumferential direction of the retaining element (8). Both first retaining members (8d) and both second retaining members (8d′) comprise an outward-protruding snap cam (8e; 8e′) and an inward-protruding snap cam (8f; 8f′). A plurality of retaining element ribs (8g) are arranged on the outer circumferential surface of the retaining element (8) and can be in sliding contact with a distal metering element inner circumferential surface (9f) provided on the inner circumferential surface of the metering element (9), in order to achieve a coaxial guidance between the retaining element (8) and the metering element (9) during the process of discharging the fixed dose from the injection device. In addition, the retaining element ribs (8g) can be used to form a guide in order to allow an elastic outward pivoting of the first retaining member (8d) and/or the second retaining member (8d′), having the first outward-protruding snap cam (8e) and/or the second outward-protruding snap cam (8e′), and the first inward-protruding snap cam (8f; visible in FIG. 2a, for example) and/or the second inward-protruding snap cam (8f′; visible in FIG. 2b, for example), or said retaining element ribs can be used for an elastic outward tensioning of the first retaining member (8d) and/or the second retaining member (8d′), having the first outward-protruding snap cam (8e) and/or the second outward-protruding snap cam (8e′) and having the first inward-protruding snap cam (8f; visible in FIG. 2a, for example) and/or the second inward-protruding snap cam (8f′; visible in FIG. 2b, for example). For this purpose, the retaining element ribs (8g) extend parallel to the first retaining member (8d) and/or the second retaining member (8d′) on the retaining element. The injection device further comprises a discharge element (6). The sleeve-like discharge element (6) has received a prestressed spring (7) in the interior which is supported at the distal end on a distal end wall of the discharge element (6) and at the proximal end on a proximal end wall of the retaining element (8). The sleeve-like discharge element (6) comprises a first recess (6a), which is formed such that the first inward-protruding snap cam (8f, visible in FIGS. 2a and 2b, for example) of the first retaining member (8d) can be received. In addition, the sleeve-like discharge element (6) comprises a second recess (6a′), which is formed such that the second inward-protruding snap cam (8f′, visible in FIGS. 2a and 2b, for example) of the second retaining member (8d′) can be received. The first recess (6a) and the second recess (6a′) of the discharge element (6) are arranged partially offset from one another in the circumferential direction and in the axial direction. The first recess (6a) and the second recess (6a′) are provided partially offset from one another such that they overlap in the circumferential direction. The discharge element (6) further comprises a plurality of first recesses (6a) that are arranged axially offset from one another and a plurality of second recesses (6a′) that are likewise arranged axially offset from one another. In addition, the discharge element (6) comprises two first recesses (6a), which are arranged offset from one another by an angle of approximately 180° in the circumferential direction of the discharge element (6), wherein the discharge element (6) provides a plurality of first recesses (6a) arranged offset axially from one another. Furthermore, the discharge element (6) comprises two second recesses (6a′), which are arranged offset from one another by an angle of approximately 180° in the circumferential direction of the discharge element (6), wherein the discharge element (6) provides a plurality of second recesses (6a′) arranged offset axially from one another. The first recess (6a) and the second recess (6a′) are arranged relative to one another at approximately a 90° angle in the circumferential direction of the discharge element (6). The sleeve-like discharge element (6) is provided radially between the prestressed spring (7) and the retaining element (8), wherein a distal and a proximal tensioning element (3; 5) are mounted on a distal region of the discharge element (6). A prestressed spring (4) is arranged between the distal and proximal tensioning elements (3: 5), wherein the prestressed spring (4) can be tensioned by an axial relative movement between the distal and proximal tensioning elements (3; 5). The spring (4) is supported between a tensioning element annular web (3a) of the distal tensioning element (3) and a tensioning element ring (5a) of the proximal tensioning element (5). The proximal tensioning element (5) comprises a proximal tensioning element tensioner (5b) and the distal tensioning element (3) has a tensioning element hook (3b). The tensioning element hook (3b) of the distal tensioning element (3) can protrude through an inner opening of the proximal tensioning element (5) and interlock with the tensioning element ring (5a) of the proximal tensioning element (5) in such a manner that the distal tensioning element (3), the spring (4) and the proximal tensioning element (5) can form an assembly unit. In the assembled state of the injection device, the proximal tensioning element ring (5a) of the proximal tensioning element (5) can strike against a retaining element stop (8h) provided on the inner circumferential surface of the retaining element (8), wherein this stopping contact can be released in order to form an acoustic signal, particularly a click sound. On the inner circumferential surface, the retaining element (8) has a retaining element groove (8j) having a proximal end face that can receive, with the proximal end face thereof, a proximal end of the proximal tensioning element snapper (5b).

FIG. 2a shows a longitudinal section of the first embodiment of the injection device in an initial position of a metering element (9), FIG. 2b showing the injection device according to FIG. 2a, wherein the injection device is rotated about the longitudinal axis B-B by 90°. The first retaining member (8d) protrudes with the first inward-protruding snap cam (8f) into the first recess (6a) of the discharge element (6). The first inward-protruding snap cam (8f) is kept engaged with the first recess (6a) of the discharge element (6) by the metering element (9) via the first inward-projecting snap cam (8e) of the first retaining member (8d), in such a manner that the first retaining member (8d) is prevented by the inward-protruding snap cam (8f) from being deflected elastically radially outward in order to disengage from the first recess (6a) of the discharge element (6). The spring (7), which is received by the discharge element (6), is supported on the distal side against the discharge element (6) and on the proximal side by the retaining element (8) and applies a spring pressure in the distal direction to the discharge element (6). A distal end face of the connecting web of two first recesses (6a) of the discharge element (6) that are offset from one another in the axial direction is in stopping contact with a proximal end face of the inward-protruding snap cam (80 of the first retaining member (8d) and prevents a movement of the discharge element (6) relative to the housing (10) in the distal direction for discharging the fixed dose. The second inward-protruding snap cam (8f′) of the second retaining member (8d′) protrudes elastically relaxedly into the second recess (6a′) of the discharge element (6). The metering element cutout (9c) of the metering element (9) is arranged above the second outward-protruding snap cam (8e′) of the second retaining member (8d′), the second inward-protruding snap cam (8f′) of the second retaining member (8d′) being disengaged from the metering element cutout (9c) of the metering element (9). A proximal end face of the proximal tensioning element ring (5a) of the proximal tensioning element (5) strikes against a distal end face of the retaining element stop (8h) of the retaining element (8), in order to tension the spring (4) between the distal tensioning element annular web (3a) of the distal tensioning element (3) and the proximal tensioning element ring (5a) of the proximal tensioning element (5). The distal tensioning element (3) is axially and rotationally fixedly connected to the carpule holder (1) via a distal tensioning element cutout (3c) and the distal tensioning element annular web (3a). The proximal tensioning element (5) is mounted axially movably on the discharge element (6), where the proximal tensioning element ring (5a) is in sliding contact with the circumferential inner surface of the retaining element (8). One proximal tensioning element snapper (5b) protrudes elastically relaxedly into the first recess (6a), and the other proximal tensioning element snapper (5b) protrudes elastically relaxedly into the second recess (6a′) of the discharge element (6).

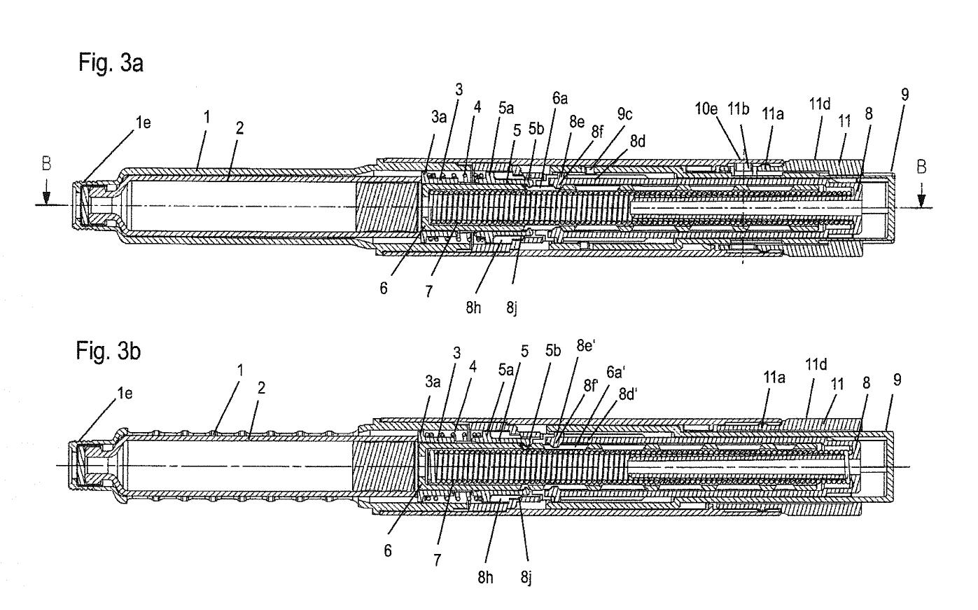

FIG. 3a shows a longitudinal section of the first embodiment of the injection device in a retracted position of the metering element (9), FIG. 3b showing the injection device according to FIG. 3a, wherein the injection device is rotated about the longitudinal axis B-B by 90°. To make the injection device ready for injection, the user rotates the setting button (11) relative to the housing (10) via the setting button handle web (11d). The setting button snap arm (11b) of the setting button (11) slides over the proximal housing groove or the proximal housing ramp (10e) of the housing (10) and prevents rotation of the setting button (11) in the opposite direction. The snapping of the setting button snap arm (11b) of the setting button (11) behind the proximal housing groove or housing ramp (10e) of the housing (10) generates an acoustic sound, in particular a click sound. The rotational movement is transmitted to the metering element (9) by the rotationally fixed connection between the setting button web (11c) of the setting element (11) and the proximal metering element groove (9a) of the metering element (9), wherein the metering element (9) is moved in the proximal direction by means of the guiding engagement or slotted guide between the metering element cam (9e) of the metering element (9) and the retaining element control cam (8c) of the retaining element (8), as can be seen in FIG. 4. At the end of the retracting movement of the metering element (9), the metering element web (9b) of the metering element (9) comes into contact with a steep flank of the housing control cam (10f) of the housing (10), this stopping contact limiting the retracting movement. As shown in FIG. 4, the metering element cam (9e) of the metering element (9) is axially aligned at the end of the retracting movement of the metering element (9) with a steep flank of the retaining element control cam (8c) of the retaining element (8) such that, due to the limitation of the retracting movement between the metering stop web (9b) of the metering element (9) and the housing control cam (10f) of the housing (10), the metering element (9) can be moved in the distal direction for discharging the fixed dose. In the retracted position of the metering element (9), the first inward-protruding snap cam (80 of the first retaining member (8d) is still kept engaged by the metering element (9) with the first cutout (6a) of the discharge element (6) via the first outward-protruding snap cam (8e) of the first retaining member (8d), wherein the metering element cutout (9c) of the metering element (9) comes into axial alignment with, and is offset in the proximal direction from, the first outward-protruding snap cam (8e) of the first retaining member (8d), due to the relative rotational movement of the metering element (9). Due to the relative rotational movement of the metering element (9), the second inward-protruding snap cam (8f′) of the second retaining member (8d′) is kept in engagement with the second recess (6a′) of the discharge element (6) via the second outward-protruding snap cam (8e′) of the metering element (9).

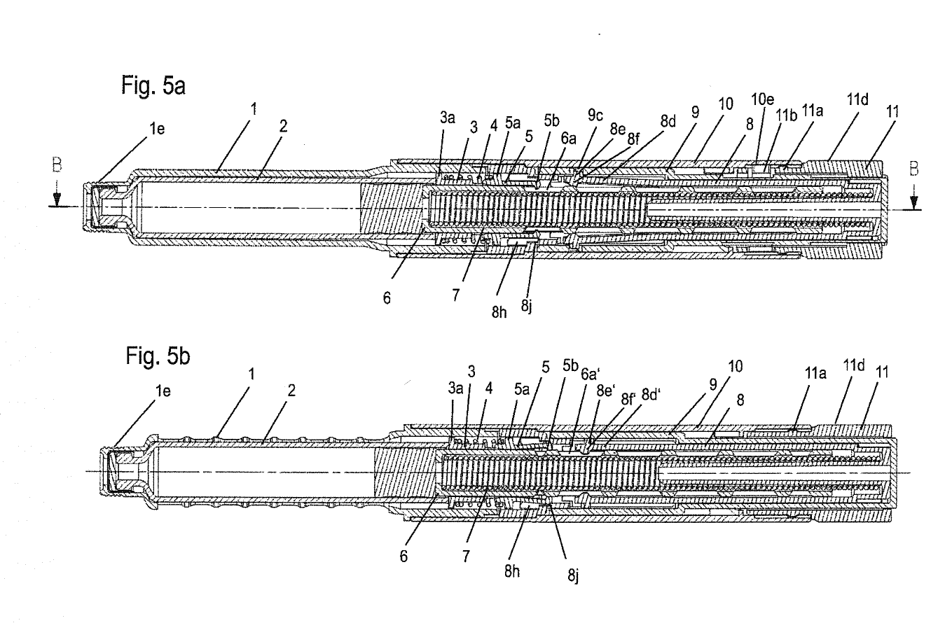

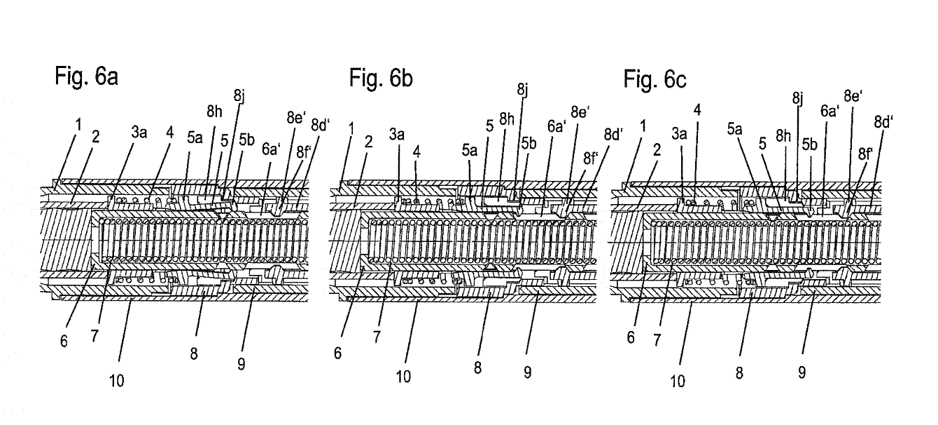

FIG. 5a shows a longitudinal section of the first embodiment of the injection device in the injection-triggered position of the metering element (9), FIG. 5b showing the injection device according to FIG. 5a, wherein the injection device is rotated about the longitudinal axis B-B by 90°. In order to trigger the injection of the fixed dose, the user actuates the metering element (9) by moving the metering element (9) axially in the distal direction relative to the retaining element (8) or the housing (10) until the proximal end of the metering element (9) strikes a proximal end face of the retaining element (8). Due to the axial movement of the metering element (9) relative to the retaining element (8), the metering element cutout (9c) of the metering element (9) moves above the first outward-protruding snap cam (8e) of the retaining element (8) such that the first outward-protruding snap cam (8e) of the retaining element (8) is deflected elastically into the metering element cutout (9c) of the metering element (9) due to the axial pressure exerted by the discharge element (6) onto the first retaining member (8d) of the retaining element (8). Thereby the first inward-protruding snap cam (8f) of the first retaining member (8d) comes out of engagement with the first recess (6a) of the discharge element (6). In addition, the stopping contact between the proximal end face of the first inward-protruding snap cam (8f) and the distal end face of the connecting web of two first recesses (6a) of the discharge element (6) that are arranged offset from one another axially is released, so that the connecting web moves in the distal direction relative to the first inward-protruding snap cam (8f). The discharge element (6) moves axially in the distal direction relative to the housing (10) during the process for discharging the fixed dose. During the discharging process, the proximal end face of the proximal tensioning element tensioner (5b) strikes against a distal end face of the connecting web of two second recesses (6a′) of the discharge element (6) arranged offset from one another in the axial direction, so that the proximal tensioning element (5) slides in the distal direction relative to the distal tensioning element (3) having the discharge element (6), and consequently, the retaining element stop (8h) comes out of stopping contact with the proximal tensioning element ring (5a), as can be seen in FIGS. 5b and 6a. Thereby the spring (4) is tensioned or further tensioned between the distal tensioning element (3) and the proximal tensioning element (5). The proximal tensioning element (5) moves axially relative to the distal tensioning element (3) until the proximal tensioning element snapper (5b) is elastically deflected radially outward into the retaining element groove (8j). The proximal end face of the proximal tensioning element snapper (5b) comes into stopping contact with the proximal end face of the retaining element groove (8j), the spring (4) remaining in the tensioned state. The connecting web provided between two second recesses (6a′) of the discharge element (6) that are arranged offset from one another in the axial direction slides along the proximal tensioning element snapper (5b), until the proximal tensioning element snapper (5b) elastically deflects into the more proximal second recess (6a′) of the discharge element (6), as shown in FIG. 6b. The distal end face of the connecting web of two second recesses (6a′) of the discharge element (6), offset from one another in the axial direction, comes into stopping contact with the proximal end face of the inward-protruding snap cam (8f′) of the second retaining member (8d′) and prevents a further movement of the discharge element (6) relative to the retaining element (8) in the distal direction for discharging the fixed dose. The discharge movement is stopped in this way. The second retaining member (8d′) of the retaining element (8) is prevented by the metering element (9) from elastically deflecting radially outward, since the metering element cutout (9c) of the metering element (9) is arranged above the first retaining member (8d) of the retaining element (8). As can be seen in FIG. 5a, the connecting web of two first recesses (6a) of the discharge element (6) that are arranged offset from one another slides along the first inward-protruding snap cam (80 of the first retaining member (8d) of the retaining element (8), until the first inward-protruding snap cam (8f) deflects elastically into the more proximal first recess (6a) of the first two recesses (6a) of the discharge element (6), wherein the first retaining element (8d) elastically relaxes. As shown in FIG. 6c, the stopping contact between the proximal end face of the proximal tensioning element snapper (5b) and the proximal end face of the retaining element groove (8j) is released by the deflection of the proximal tensioning element snapper (5b) into the more proximal second recess (6a) of the discharge element (6). The spring force of the spring (4) acts on the proximal tensioning element (5) in such a manner that the proximal tensioning element (5) is moved axially relative to the retaining element (8) in the proximal direction until the proximal tensioning element ring (5a) of the proximal tensioning element (5) strikes against the retaining element stop (8h) of the retaining element (8). This stopping contact triggers an acoustic signal, in particular a click sound. The end of injection of the fixed dose from the injection device can therefore be indicated.

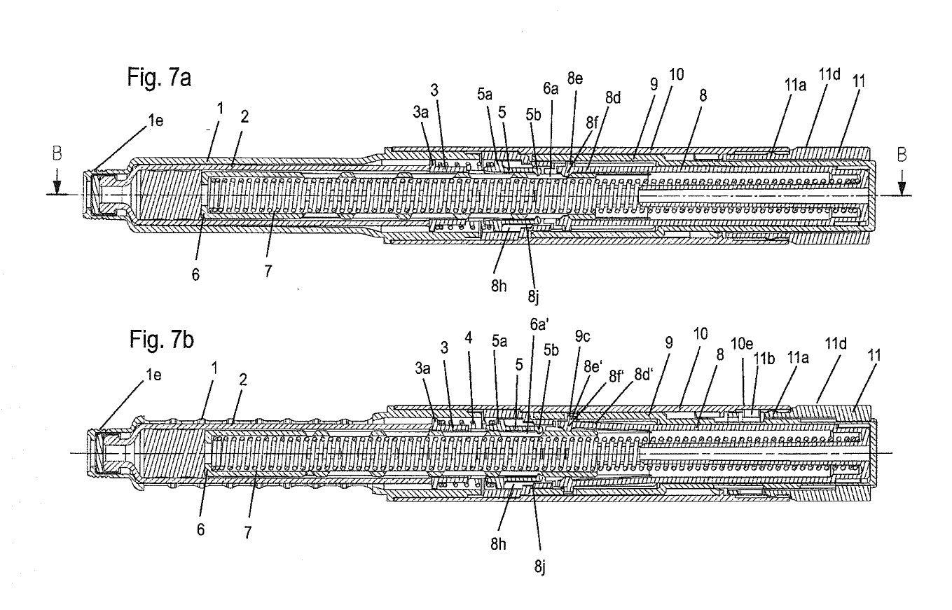

FIG. 7a shows a longitudinal section of the first embodiment of the injection device in the completely discharged position of a metering element (9), FIG. 7b showing the injection device according to FIG. 7a, wherein the injection device is rotated about the longitudinal axis B-B by 90°. The positions of the individual parts of the injection device according to FIG. 7a correspond to the positions of the individual parts of the injection device according to FIG. 2a, except that the discharge element (6) is displaced so far axially in the distal direction in relation to the retaining element (8) that the first inward-protruding snap cam (8e) of the retaining element (8) is located in the proximally first recess (6a) of the discharge element (6). The first retaining member (8d) of the retaining element (8) is prevented by the metering element (9) from deflecting radially outward. The second outward-protruding snap cam (8e′) of the second retaining member (8d′) protrudes into the metering element cutout (9c) of the metering element (9). Because no additional second recess (6a′) of the discharge element (6), namely a second recess (6a′) of the discharge element (6) in the proximal direction of the first recess (6a) of the discharge element (6), is provided, the second retaining member (8d′) of the retaining element (8) is prevented from deflecting radially inward. The second outward-protruding snap cam (8e′) of the second retaining member (8d′) protrudes into the metering element cutout (9c) of the metering element (9) and prevents further setting or metering of a fixed dose. The final fixed dose has been discharged from the injection device.

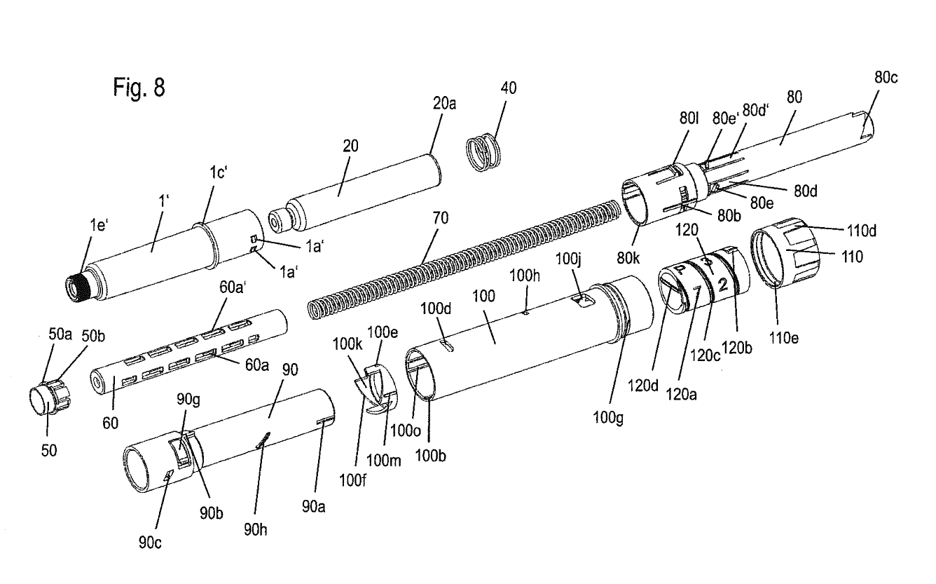

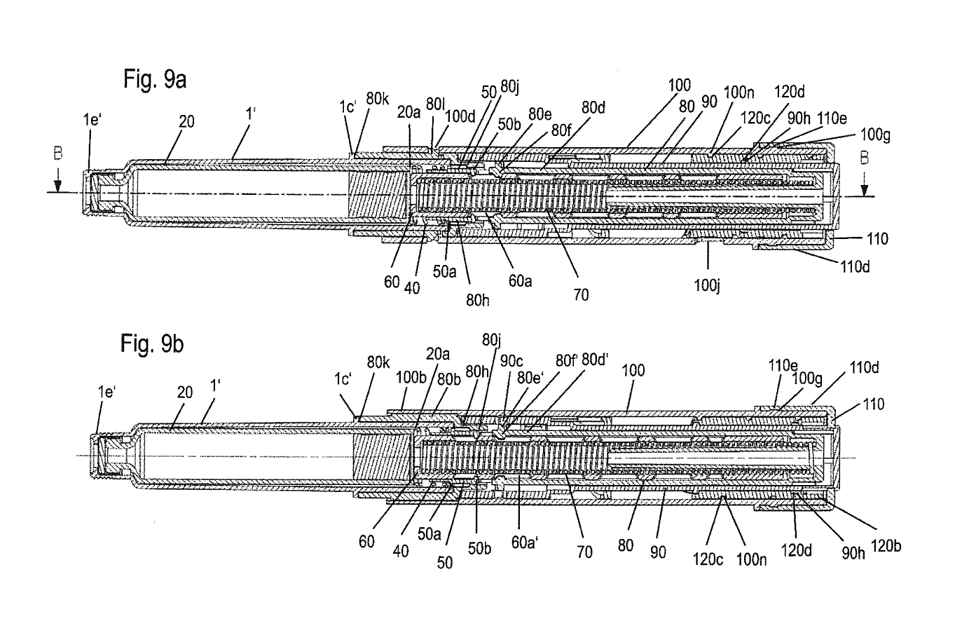

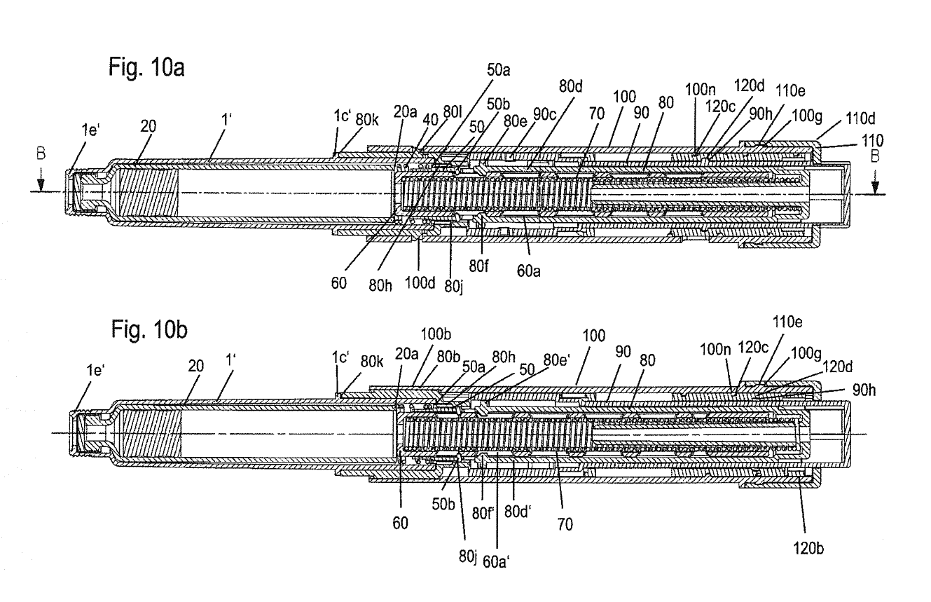

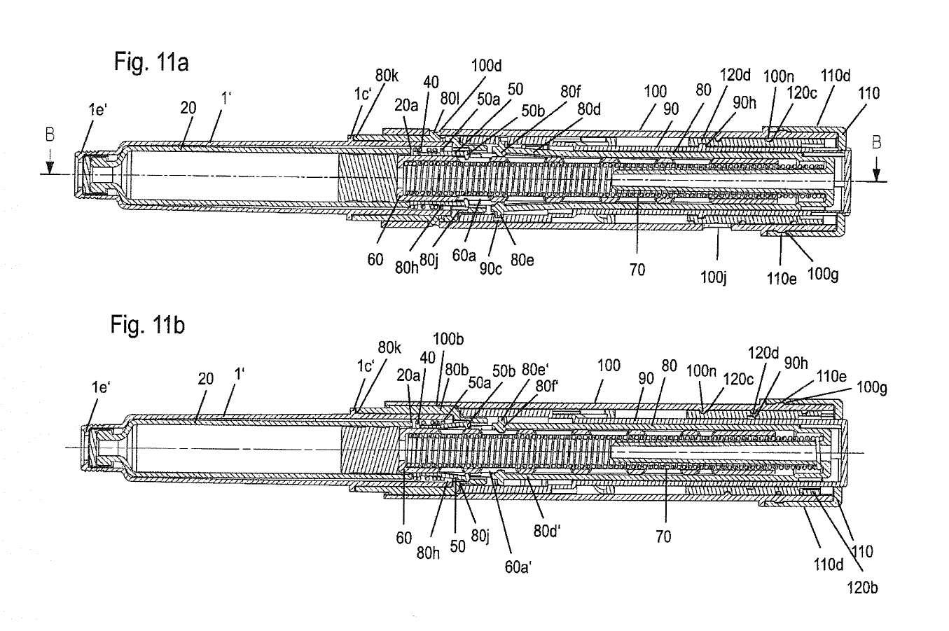

FIG. 8 shows an exploded view of a second embodiment of an injection device according to the invention. The injection device comprises a carpule (20), which is received by a carpule holder (1′). The carpule holder (1′) is connected rotationally fixedly and axially fixedly via at least one carpule holder tab (1a′), two in this embodiment, to at least one retaining element cutout (not visible), two in this embodiment, arranged on the inner circumferential surface of a retaining element (80). A carpule holder stop (1c′) can strike axially against a distal retaining element edge (80k) of the retaining element (80). The distal end of the carpule holder (1′) has a needle-connecting element (1e′) that is provided for detachably mounting an injection needle. The injection device further comprises a metering element (90), which is provided radially between the housing (100) and the retaining element (80). A retaining element web (80b) is provided on the retaining element (80) for detachable connection to a corresponding distal housing groove (100b). The retaining element (80) is axially fixedly connected via a distal retaining element snap arm (80l) to a proximal housing tab cutout (100d) of the housing (100). A setting button (110), which is axially fixedly connected via a setting button annular groove (110e) of the setting button (110) to an annular housing web (100g) of the housing (100), is additionally provided. A setting button handle web (110d) for better gripping of the setting button (110) by the user is provided on the setting button (110). The setting button (110) comprises, on the inner circumferential surface thereof, a setting button web (not visible), which forms a rotationally fixed connection to a proximal metering element groove (90a) provided on the outer circumferential surface of the metering element (90). The metering element (90) comprises a metering element snap arm (90g), which can cooperate with a housing ramp (100o) provided on the inner circumferential surface of the housing (100) in such a manner that the metering element (90) can rotate in only one rotational direction relative to the housing (100). This cooperation between the metering element snap arm (90g) and the distal housing ramp (100o) can produce an acoustic sound, in particular a click sound. The metering element (90) comprises, on the inner circumferential surface thereof, a metering element cam (90e; visible in FIG. 10c) may serve as a guide element, which can be guided along a sawtooth-shaped retaining element curve (80c) of the retaining element (80) during rotation of the metering element (90), and the retaining element curve (80c) may serve as a counter-guide element. When the metering element (90) rotates relative to the retaining element (80), this guiding engagement or the slotted guide between the metering element cam (90e; visible in FIG. 10c) and the retaining element curve (80c) has the effect that the metering element (90) can be moved axially in the proximal direction relative to the housing (100). The metering element (90) has a metering element web (90b), which is in a guiding engagement with a sawtooth-shaped housing control cam (100f) of a housing ring (100k) or in the slotted guide thereof. The housing ring (100k) is axially fixedly and rotationally fixedly connected to the housing (100) via a housing ring tab (100l); visible in FIG. 10c) and a housing ring web (100m) with a housing tab cutout (100h) and a housing ring groove (not visible). The metering element web (90b) of the metering element (90) and the housing control cam (100f) of the housing ring (100k) can cooperate in such a manner that more than one fixed dose to be administered can be set upon rotation of the metering element (90) relative to the housing (100). A metering element cutout (90c) is provided in the metering element (90) and is designed such that an outward-protruding retaining element snap cam (80e; 80e′), which is mounted on a retaining member (80d; 80d′) of the retaining element (80), can protrude into this metering element cutout (90c). The metering element (90) comprises two metering element cutouts (90c), which are offset relative to one another by approximately 180° in the circumferential direction of the metering element (90). The retaining member (80d; 80d′) can be formed as a snap arm. The injection device comprises a first retaining member (80d) and a second retaining member (80d′) or snap arm, wherein the first retaining member (80d) and the second retaining member (80d′) comprise a first outward-protruding snap cam (80e) and a second outward-protruding snap cam (80e′) and a first inward-protruding snap cam (80f; visible in FIGS. 9a and 9b, for example) and a second inward-protruding snap cam (80f′; visible in FIGS. 9a and 9b, for example). The first retaining member (80d) and the second retaining member (80d′) are formed as elastic pivot arms in the retaining element (80). The first outward-protruding snap cam (80e) and the second outward-protruding-snap cam (80e′) and the first inward-protruding snap cam (80f) and the second inward-protruding snap cam (80f′) are also arranged at approximately equal axial height on the retaining element (80). The first inward-protruding snap cam (80f) is arranged on the first retaining member (80d) approximately diametrically opposite the first outward-protruding snap cam (80e), while the second inward-protruding snap cam (80f′) is provided on the second retaining member (80d′) approximately diametrically opposite the second outward-protruding snap cam (80e′). The first retaining member (80d) and the second retaining member (80d′) are arranged relative to one another at approximately a 90° angle in the circumferential direction of the retaining element (80). In addition, two first retaining members (80d) and two second retaining members (80d′) are provided on the retaining element (80), wherein the two first retaining members (80d) are arranged at approximately a 180° angle relative to one another in the circumferential direction of the retaining element (80), and the two second retaining members (80d′) are arranged at approximately a 180° angle relative to one another in the circumferential direction of the retaining element (80). Both first retaining members (80d) and both second retaining members (80d′) each comprise an outward-protruding snap cam (80e; 80e′) and an inward-protruding snap cam (80f; 80f′). The injection device further comprises a discharge element (60), which has received a prestressed spring (70). A first recess (60a) and a second recess (60a′) are provided in the discharge element. The first recess (60a) is suitable for receiving the first inward-protruding snap cam (80f; visible in FIG. 9a) and the second recess (60a′) is suitable for the second inward-protruding snap cam (80f′; visible in FIG. 9b). The first recess (60a) and the second recess (60a′) of the discharge element (60) are arranged partially offset from one another in the circumferential direction and in the axial direction. In addition, the first recess (60a) and the second recess (60a′) are provided partially offset from one another in the discharge element (60) such that they overlap in the circumferential direction. The discharge element (60) further comprises a plurality of first recesses (60a) that are arranged axially offset from one another in a plurality of second recesses (60a′) that are likewise arranged axially offset from one another. The first recess (60a) and the second recess (60a′) are arranged relative to one another at approximately a 90° angle in the circumferential direction of the discharge element (60). In addition, the discharge element (60) comprises two first recesses (60a), which are arranged offset from one another by an angle of approximately 180° in the circumferential direction of the discharge element (60), wherein the discharge element (60) provides a plurality of first recesses (60a) arranged offset axially from one another. The discharge element (60) further comprises two second recesses (60a′), which are arranged offset from one another by an angle of approximately 180° in the circumferential direction of the discharge element (60), wherein the discharge element (60) provides a plurality of second recesses (60a′) arranged offset axially from one another. A proximal tensioning element (50) having a proximal tensioning element ring (50a) and a proximal tensioning element snapper (50b) is mounted axially movably on the discharge element (60). A spring (40), which can be tensioned by an axial displacement of the proximal tensioning element (50) in the distal direction relative to the housing (100), is provided between the proximal tensioning element ring (50a) and a carpule edge (20a). The proximal tensioning element ring (50a) provided on the proximal tensioning element (50) can strike against a retaining element stop (80h; visible in FIG. 9a or 9b, for example) provided on the retaining element (80) in order to generate an acoustic signal, in particular a click sound. The inner circumferential surface of the retaining element (80) additionally has a retaining element groove (80j; visible in FIG. 9a or 9b, for example), which can receive the distal end of the proximal tensioning element snapper (50b). The injection device further comprises a display sleeve (120) having at least one display digit (120a) for displaying a discharged and/or a still available defined dose and/or a metered defined dose. The display digit (120a) of the display sleeve (120) can be visible through a housing cutout (100j) of the housing (100). A marking applied to the display sleeve (120) and/or an intermediate space between two display digits (120a) provided on the display sleeve (120), or two markings can be visible through the cutout (100j) of the housing (100) in order to display a discharged and/or a still available defined dose and/or a metered defined dose. On the outer circumferential surface, the metering element (90) has a metering element external thread (90h), which can be in threaded engagement with a display sleeve internal thread (120d) provided on the inner circumferential surface of the display sleeve (120). On the circumferential outer surface, the display sleeve (120) has a display sleeve external thread (120c), which can be in threaded engagement with a housing internal thread (100n; visible in FIG. 12, for example) positioned on the inner circumferential surface of the housing. The two threaded connections of the display sleeve (120) are formed in different directions and with different pitches, the thread pitch of the threaded connection between the display sleeve (120) and the housing (100) being formed less steeply than the thread pitch of the threaded connection between the display sleeve (120) and the metering element (90). A housing internal thread start (100p; visible in FIG. 12, for example) is provided at the proximal end of the housing internal thread (100n), and the proximal end of the display sleeve external thread (120c) has a display sleeve external thread end (120e), wherein the housing internal thread start (100p) and the display sleeve external thread end (120e) can be in stopping contact in order to prevent the setting and/or metering of a further fixed dose. The display sleeve (120) further comprises a display sleeve snap arm (120b), which can interact with a proximal housing groove or proximal housing ramp (not visible) in such a manner that the display sleeve (120) can be rotated in only one rotational direction. The display sleeve can be moved axially relative to the housing (100) only in the distal direction. This interaction can trigger an acoustic signal, in particular a click sound.

FIG. 9a shows a longitudinal section of the second embodiment of the injection device in an initial position of a metering element (90), FIG. 9b showing the injection device according to FIG. 9a, wherein the injection device is rotated about the longitudinal axis B-B by 90°. The first outward-protruding snap cam (80e) of the first retaining member (80d) is in stopping contact with the circumferential inner surface of the metering element (90) in such a manner that the first inward-protruding snap cam (80f) of the first retaining member (80d) protrudes into the first recess (60a) of the discharge element (60) and strikes against the distal end face of the connecting web of two first recesses (60a) offset from one another in the axial direction in such a manner that the first inward-protruding snap cam (80f) does not become disengaged from the first recess (60a) of the discharge element (60). The circumferential surface of the metering element (90) lies above the first outward-protruding snap cam (80e) of the first retaining member (80d) of the first retaining element (80), in order to prevent the first inward-protruding snap cam (80f) of the first retaining member (80d) of the retaining element (80) from being able to disengage from the first recess (60a) of the discharge element (60). The prestressed spring (70), which is provided in the discharge element (60), is supported on the distal side against the discharge element (60) and on the proximal side by the retaining element, and applies a spring pressure in the distal direction to the discharge element (60). The stopping contact between the first inward-protruding snap cam (80f) of the first retaining member (80d) and the distal end face of the connecting web of two first recesses arranged offset in the axial direction causes an inhibition of a movement of the discharge element (60) in the distal direction relative to the housing (10). The second inward-protruding snap cam (80f′) of the second retaining member (80d′) is elastically relaxed in the second recess (60a′) of the discharge element (60). The metering element cutout (90c) of the metering element (90) lies above the second outward-protruding snap cam (80e′) of the second retaining member (80d′), the second outward-protruding snap cam (80e′) of the second retaining member (80d′) being disengaged from the metering element cutout (90c) of the metering element (90). A proximal end face of the proximal tensioning element snapper (50b) of the proximal tensioning element (50) has a stopping contact with a distal end face of the connecting web of two recesses (60a) of the discharge element (60) that are arranged offset from one another in the axial direction. Thereby the spring (40) is held prestressed between the carpule edge (20a) and the proximal tensioning element ring (50a). The proximal tensioning element (50) is movably mounted on the discharge element (60). One proximal tensioning element snapper (50b) elastically protrudes relaxedly into the first recess (60a), and the other proximal tensioning element snapper (50b) elastically protrudes relaxedly into the second recess (60a′) of the discharge element.

FIG. 10a shows a longitudinal section of the second embodiment of the injection device in a retracted position of the metering element (90), FIG. 10b showing the injection device according to FIG. 10a, wherein the injection device is rotated about the longitudinal axis B-B by 90°. To make the injection device ready, the user rotates the setting button (110) relative to the housing (100) with the aid of the setting button web (110d). The rotationally fixed connection between the proximal metering element groove (90a) of the metering element (90) and the setting button web of the setting button (110) causes the rotational movement of the setting button (110) to be transmitted to the metering element (90). Due to the guiding engagement or the slotted guide between the metering element cam (90e) of the metering element (90) and the retaining element control cam (80c) of the retaining element (80), the metering element (90) moves in the proximal direction relative to the housing (100), as shown in FIG. 10c. It can also be seen in FIG. 10c that, at the end of the retracting movement of the metering element (90), the metering element web (90b) of the metering element (90) is in stopping contact with a steep flank of the housing control cam (100f) of the housing ring (100k). This stopping contact causes a restriction of the retracting movement such that no further fixed dose can be metered. In order to set or meter an additional fixed dose, the already set or metered fixed dose must first be discharged by moving the metering element (90) axially in the distal direction relative to the housing (100). For this purpose, the metering element cam (90e) of the metering element (90) is axially aligned with a steep flank of the retaining element control cam (80c) of the retaining element (80) in the retracted position of the metering element (90). In addition, the metering element cutout (90c) of the metering element (90) is axially aligned with the first outward-protruding snap cam (80e) of the first retaining member (80d). The second inward-protruding snap cam (80 f′) of the second retaining member (80d′) is engaged with the second recess (60a′) of the discharge element (60), with the aid of the second outward-protruding snap cam (80e′) of the second retaining member (80d′). By rotating the metering element (90) relative to the housing (100) during the metering movement of the injection device, the display sleeve is screwed axially in the distal direction (120) via the threaded connection between the housing internal thread (100n) of the housing and the display sleeve external thread (120c) of the display sleeve (120), and via the threaded connection between the display sleeve internal thread (120d) of the display sleeve (120) and the metering element external thread (90h) of the metering element (90). The helically arranged display digits (120a) of the display sleeve (120) are visible through the housing cutout (100j) of the housing (100), the two threaded connections and the display digits (120a) of the display sleeve (120) being designed such that, in the initial position of the metering element (90), a different display digit (120a) can be seen than in the retracted position of the metering element (90), or an intermediate space between two display digits (120a) or a marking can be seen through the display cutout (100j) of the display (100) in the initial position. The display sleeve snap arm (120b) of the display sleeve (120) cooperates with the proximal housing groove or the proximal housing ramp of the housing (100) in such a manner that the display sleeve (120) can only rotate in one direction.

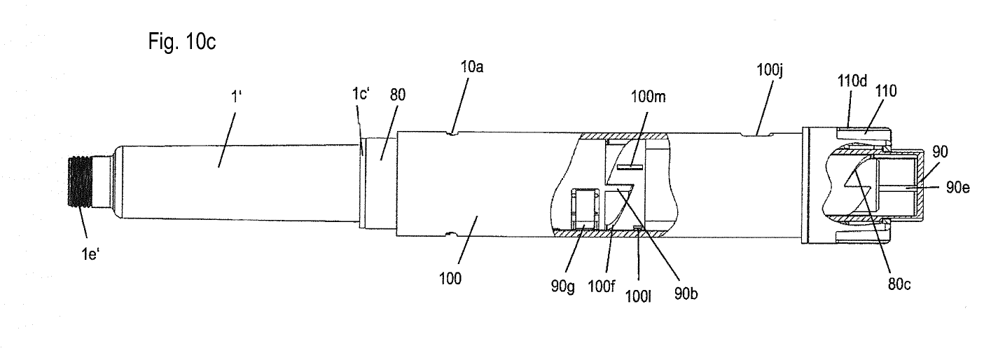

FIG. 11a shows a longitudinal section of the second embodiment of the injection device in an injection-triggered position of the metering element (90), FIG. 11b showing the injection device according to FIG. 11a, wherein the injection device is rotated about the longitudinal axis B-B by 90°. In order to trigger the fixed dose, the metering element (90) is moved in the distal direction relative to the retaining element (80) or the housing (100) until the metering element (90) comes into stopping contact with the retaining element (80) fixed in relation to the housing. The metering element (90) moves axially relative to the retaining element (80) or the housing (100) until the metering element cutout (90c) of the metering element (90) comes into the area of the first outward-protruding snap cam (80e) of the first retaining member (80d), so that the first outward-protruding snap cam (80e) of the first retaining member (80d) can deflect elastically into the metering element cutout (90c) of the metering element (90) and release the discharge element (60). The axial movement of the discharge element (60) relative to the housing (100) or the retaining element (80) causes the fixed dose to be discharged from the injection device, the relative movement being stopped by a stopping contact between a proximal end face of the second inward-projecting snap cam (80f′) of the second retaining member (80d′) and a distal end face of a connecting web of two axially offset second recesses (60a′) of the discharge element (60). The metering element (90) prevents an elastic radially outward deflection of the second inward-protruding snap cam (80f′) of the second retaining member (80d′) out of the second recess (60a′) of the discharge element (60). During the discharge movement, the connecting web of two first recesses (60a) of the discharge element (60) that are arranged axially offset from one another slides along the first inward-protruding snap cam (80f) of the first retaining member (80d) of the retaining element (80), until the first inward-protruding snap cam (80f) deflects elastically into a first recess (60a) of the discharge element (60). During the discharge movement of the fixed dose from the injection device, the spring (40) is tensioned or further pretensioned by means of the stopping contact between the proximal end face of the proximal tensioning element snapper (50b) and the distal end face of the connecting web of two second recesses (60a′) of the discharge element (60) that are arranged axially offset from one another, and by means of the axial relative movement between the discharge element (60) and the carpule (20) or the retaining element (80) or the housing (100), until the proximal tensioning element snapper (50b), or at least a part of the proximal tensioning element snapper (50b), is elastically deflected outward into the retaining element groove (80j) of the retaining element. This stopping contact causes the pretensioned spring (40) to halt. During further axial relative movement of the discharge element (60), the proximal tensioning element snapper (50b) slides over the connecting web of two second recesses (60a′) of the discharge element (60) arranged axially offset from one another, until the proximal tensioning element snapper (50b) elastically deflects radially inwardly into the proximally arranged second recess (60a′) of the two second recesses (60a′) of the discharge element (60) that are arranged axially offset from one another. Thereby the spring force of the spring (40) acts on the proximal tensioning element (50b) such that the proximal tensioning element ring (50a) of the proximal tensioning element (50b) stops against the retaining element stop (80h) of the retaining element (80) and produces an acoustic sound, in particular a click sound. Therefore, the end of injection of the fixed dose from the injection device can be indicated. During the discharge movement of the injection device, axial driving is exerted, by means of the axial displacement of the metering element (90) relative to the housing (100), onto the threaded connection between the housing internal thread (100n) of the housing (100) and the display sleeve external thread (120c) of the display sleeve (120) and onto the threaded connection between the display sleeve internal thread (120d) of the display sleeve (120) and the metering element external thread (90h) of the metering element (90), so that the display sleeve (120) is screwed axially in the distal direction. The movement of the display sleeve (120), in particular the display sleeve digits (120a) and/or the intermediate spaces between the displaced sleeve digits (120a) and/or markings, can be viewed through the housing cutout (100j) of the housing (100). The display sleeve snap arm (120b) of the display sleeve (120) moves over the housing groove or the proximal housing ramp of the housing (100), so that the display sleeve (120) can be prevented from screwing back in the proximal direction relative to the housing (100).

FIG. 12 shows a detailed view of the proximal end of the injection device of the second embodiment in a completely discharged position of the metering element (90), wherein a part of the housing (100) and a part of the setting button (110) are cut away so that the interior of the injection device is visible. After the final dose has been discharged from the injection device, the housing internal thread start (100p) of the housing internal thread (100n) is arranged in the vicinity of the display sleeve external thread end (120e) of the display sleeve external thread (120c) in such a manner that the housing internal thread start (100p) strikes against the display sleeve external thread end (120e) when an additional fixed dose is being set or metered. Consequently, no additional or at least no additional complete fixed dose can be metered via the rotationally fixed connection between the metering element (90) and the setting button (120) and via the two threaded connections between the housing (100) and the display sleeve (120) and between the display sleeve (120) and the metering element (90).

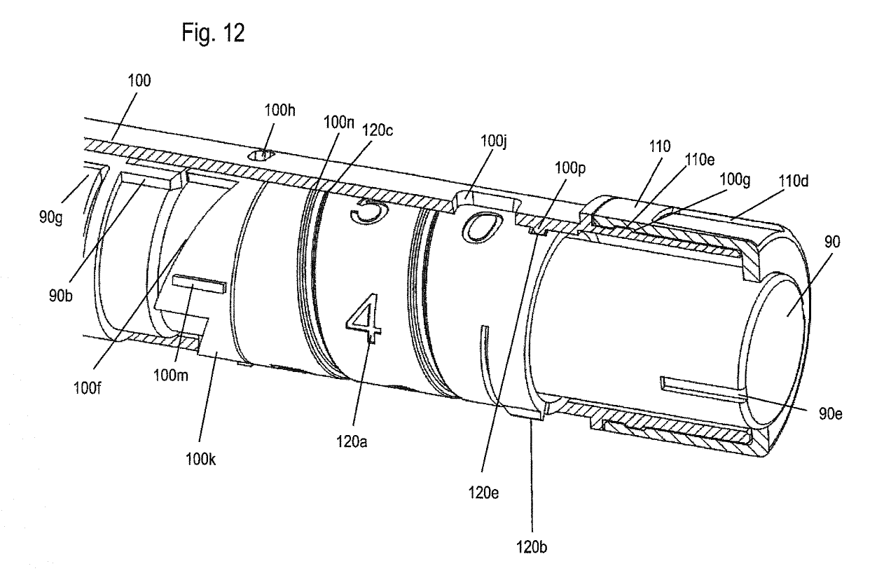

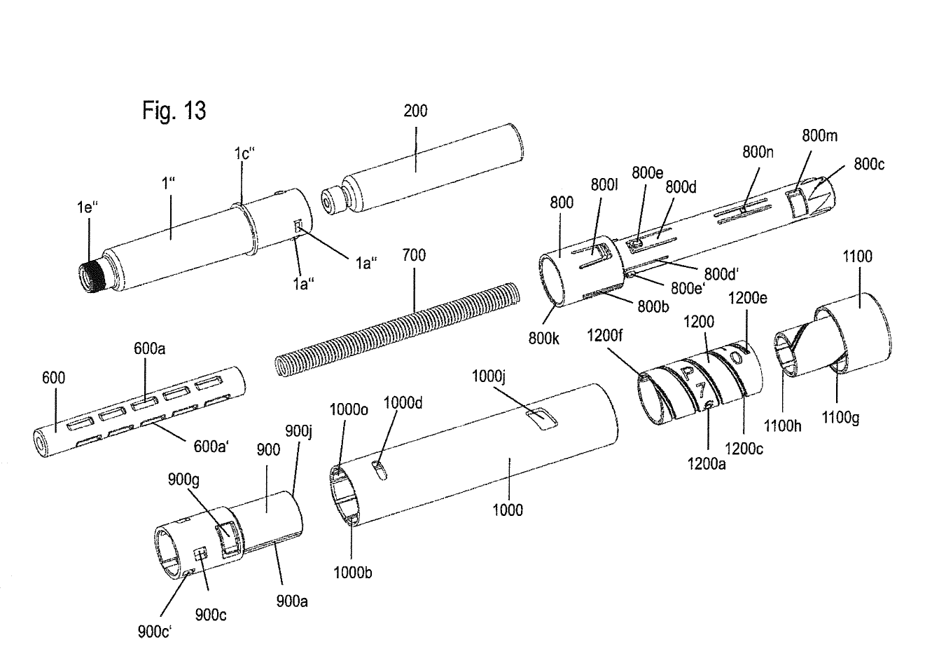

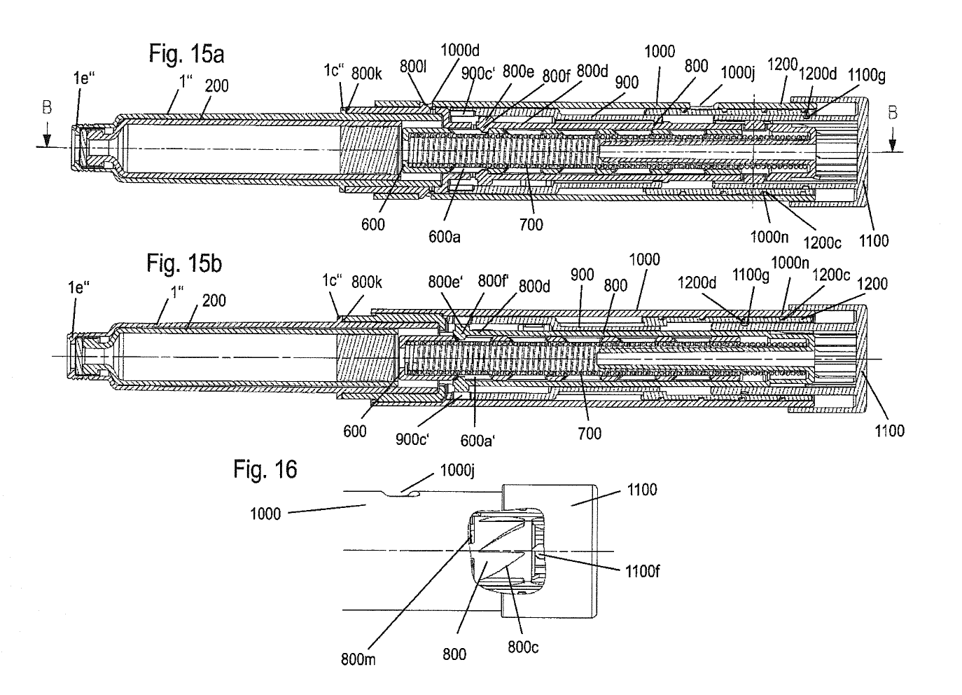

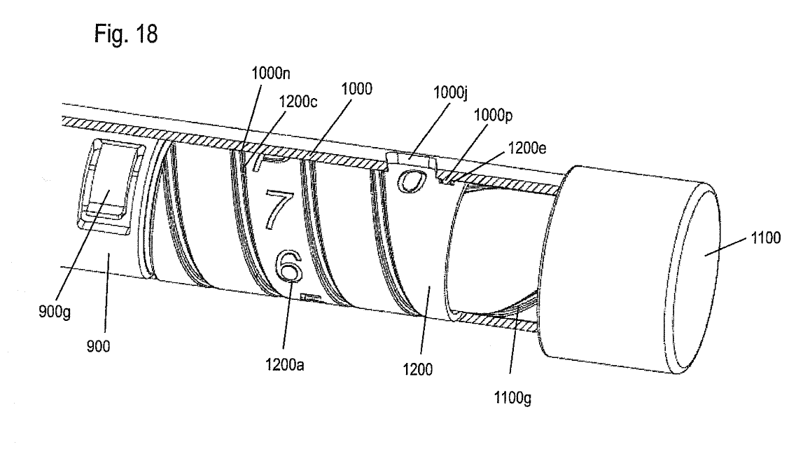

FIG. 13 shows an exploded view of a third embodiment of the injection device according to the invention. A carpule (200) is received by a carpule holder (1″), wherein the carpule holder (1″) is axially fixed and rotationally fixed via at least one carpule holder tab (1a″), two in the present embodiment, to at least one retaining element cutout (not visible), two in the present embodiment, provided on the inner circumferential surface of a retaining element (800). A distal retaining element edge (800k) can abut axially against a carpule holder stop (1c″). A needle-connecting element (1e″) for releasably mounting an injection needle is provided on the distal end of the carpule holder (1″). The injection device further comprises a metering element (900), the metering element (900) being provided radially between a housing (1000) and the retaining element (800). The retaining element (800) is rotationally fixedly connected with the aid of a retaining element web (800b) on the retaining element (800) to the housing (1000) via a distal housing groove (1000b). The axially fixed connection between the retaining element (800) and the housing (1000) is accomplished via a connection between a distal retaining element snap arm (8001) and a proximal housing cutout (1000d). The injection device further comprises a setting button (1100), which is connected to the housing (1000) via threaded connections. One threaded connection comprises a setting button external thread (1100g) of the setting button (1100) and a display sleeve internal thread (1200d, visible in FIG. 14a or 14b, for example) of a display sleeve (1200) of the injection device. On the circumferential inner surface of the setting button (1100), a setting button longitudinal groove or a setting button ramp (1100h) is provided, which cooperates with a proximal retaining element snap arm (800m) of the retaining element (800) in such a manner that the setting button (1100) is rotatable in only one direction relative to the retaining element (800). The cooperation of the proximal retaining element snap arm (800m) of the retaining element (800) with the setting button longitudinal groove or the setting button ramp (1100h) of the setting button (1100) can produce an acoustic sound, in particular a click sound. The setting button (1100) comprises, on the inner circumferential surface thereof, a setting button cam (1100f; visible in FIG. 16), which can be guided along a sawtooth-shaped retaining element curve (800c) of the retaining element (800) during rotation of the setting button (1100) relative to the housing (1000). When the setting button (1100) rotates relative to the retaining element (800), this guiding engagement or the slotted guide between the setting button cam (1100f; visible in FIG. 16) and the retaining element curve (800c) has the effect that the setting button (1100) can be moved axially in the proximal direction relative to the housing (1000) and therefore the setting button cam (1100f) and the retaining element curve (800c) may serve as a guide element and a counter-guide element, respectively, in a guiding engagement or a slotted guide engagement. The metering element (900) has a first metering element cutout (900c) and a second metering element cutout (900c′), which are designed such that a first outward-protruding retaining element snap cam (800e) of a first retaining member (800d) can protrude into the first metering element cutout (900c) and a second outward-protruding retaining element snap cam (800e′) of a second retaining member (800d′) can protrude into the second metering element cutout (900c′). The first retaining member (800d) and the second retaining member (800d′) can be constructed as a snap arm or snap tongue and are arranged offset axially from one another on the retaining element (800). The first retaining member (800d) and the second retaining member (800d′) are arranged relative to one another at approximately a 90° angle in the circumferential direction of the retaining element (800). The first inward-protruding snap cam (800f; visible in FIG. 14a, for example) is arranged on the first retaining member (800d) approximately diametrically opposite the first outward-protruding snap cam (800e), while the second inward-protruding snap cam (800f′; visible in FIG. 14b, for example) is provided on the second retaining member (800d′) approximately diametrically opposite the second outward-protruding snap cam (800e′). In addition, two first retaining members (800d) and two second retaining members (800d′) are provided on the retaining element (800), wherein the two first retaining members (800d) are arranged at approximately a 180° angle relative to one another in the circumferential direction of the retaining element (800), and the two second retaining members (800d′) are arranged at approximately a 180° angle relative to one another in the circumferential direction of the retaining element (800). Both first retaining members (800d) and both second retaining members (800d′) each comprise an outward-protruding snap cam (800e; 800e′) and an inward-protruding snap cam (800f; 800f′). The first metering element cutout (900c) and the second metering element cutout (900c′) of the metering element (900) are arranged offset from one another in the circumferential direction and in the axial direction. The first metering element cutout (900c) and the second metering element cutout (900c′) are arranged relative to one another at approximately a 45° angle in the circumferential direction of the metering element (900). In addition, the four first metering element cutouts (900c) and the four second metering element cutouts (900c′) are respectively arranged at approximately a 90° angle in the circumferential direction of the metering element (900). The injection device further comprises a discharge element (600), which has received a prestressed spring (700). A first recess (600a) and a second recess (600a′) are provided in the discharge element (600). The first recess (600a) and the second recess (600a′) of the discharge element (600) are arranged offset from one another in the circumferential direction, wherein a plurality of first recesses (600a) and a plurality of second recesses (600a′) are provided, respectively arranged offset from one another in the axial direction. The first recess (600a) and the second recess (600a′) are arranged relative to one another at approximately a 90° angle in the circumferential direction of the discharge element (600). The first recess (600a) is suitable for receiving the first inward-protruding snap cam (800f; visible in FIG. 14a) and the second recess (600a′) is suitable for the second inward-protruding snap cam (800f′; visible in FIG. 14b). In addition, the discharge element (600) comprises two first recesses (600a), which are arranged offset from one another by an angle of approximately 180° in the circumferential direction of the discharge element (600), wherein the discharge element (600) provides a plurality of first recesses (600a) arranged offset axially from one another. The discharge element (600) further comprises two second recesses (600a′), which are arranged offset from one another by an angle of approximately 180° in the circumferential direction of the discharge element (600), wherein the discharge element (600) provides a plurality of second recesses (600a′) arranged offset axially from one another. The injection device further comprises a display sleeve (1200) having at least one display digit (1200a) and/or a marking and/or an internal space for indicating a discharged and/or a still available defined dose. The display digit (1200a) of the display sleeve (1200) can be visible through a housing cutout (1000j) of the housing (1000). In addition, a marking provided on the display sleeve (1200) and/or an intermediate space between two display digits (1200a) or markings for indicating a discharged and/or a still available fixed dose can be visible through the housing cutout (1000j) of the housing (1000). The display sleeve (1200) comprises the display sleeve internal thread (1200d; visible in FIGS. 14a and 14b, for example), which is in a threaded engagement with the setting button external thread (1100g) of the setting button. The display sleeve (1200) further comprises a display sleeve external thread (1200c), which is in threaded engagement with a housing internal thread (1000n; visible in FIGS. 14a and 14b, for example). The thread pitch of the threaded connection between the display sleeve (1200) and the setting button (1100) is steeper than the thread pitch of the threaded connection between the display sleeve (1200) and the housing (1100). The pitch of the retaining element curve (800c) of the retaining element (800) is equal to the pitch of the setting button external thread (1100g) of the setting button (1100). At the proximal end of the housing internal thread (1000n; visible in FIG. 18, for example) a housing internal thread start (1000p; visible in FIG. 18, for example) is provided, and the proximal end of the display sleeve external thread (1200c) has a display sleeve external thread end (1200e: visible in FIG. 18, for example), wherein the housing internal thread start (1000p; visible in FIG. 18, for example) and the display sleeve external thread end (1200e; visible in FIG. 18, for example) can come into stopping contact in order to prevent discharging of an additional fixed dose. The display sleeve (1200) additionally has, on the inner circumferential surface thereof, a display sleeve web (1200f), which is engaged with a proximal metering element groove (900a) for a rotationally fixed connection. A rotation of the display sleeve (1200) thus causes a rotation of the metering element (900), wherein the cooperation of the metering element snap arm (900g) on the metering element (900) with a distal housing ramp (1000o) on the housing (1000) during rotation has the effect that the metering element (900) can rotate in only one direction. To prevent an axial displacement of the metering element (900) relative to the housing (1000), a retaining element cam (800n), which abuts against a proximal metering element edge (900j) of the metering element, is provided on the retaining element (800). For the purpose of a simpler assembly of the injection device, the retaining element cam (800n) is provided via a spring web on the retaining element (800).

FIG. 14a shows a longitudinal section of the third embodiment of the injection device in an initial position of a setting button (1100), FIG. 14b showing the injection device according to FIG. 14a, wherein the injection device is rotated about the longitudinal axis B-B by 90°. The first outward-protruding snap cam (800e) of the first retaining member (800d) of the retaining element (800) is in stopping contact with the inner circumferential surface of the metering element (900), while the first inwardly-protruding snap cam (800f) of the first retaining member (800d) protrudes into the first recess (600a) of the discharge element (600) and abuts against a distal end face of the connecting web of two first recesses (600a) arranged axially offset from one another. The first inwardly-protruding snap cam (800f) is engaged with the first recess (600a) of the discharge element (600). The discharge element (600), to which a force in the distal direction is applied by the prestressed spring (700), is prevented from moving axially in the distal direction relative to the housing (1000) by the stopping contact between the first inward-protruding snap cam (800f) and the connecting web of two recesses (600a) of the discharge element (600) that are arranged axially offset from one another. The metering element cutout (900c) of the metering element (900) lies above the second outward-protruding snap cam (800e′) of the second retaining member (800d′), the second outward-protruding snap cam (800e′) of the second retaining member (800d′) being disengaged from the metering element cutout (900c) of the metering element (900). The second inward-protruding snap cam (800f′) of the second retaining member (800d′) elastically protrudes relaxedly into the second recess (600a′) of the discharge element (600).

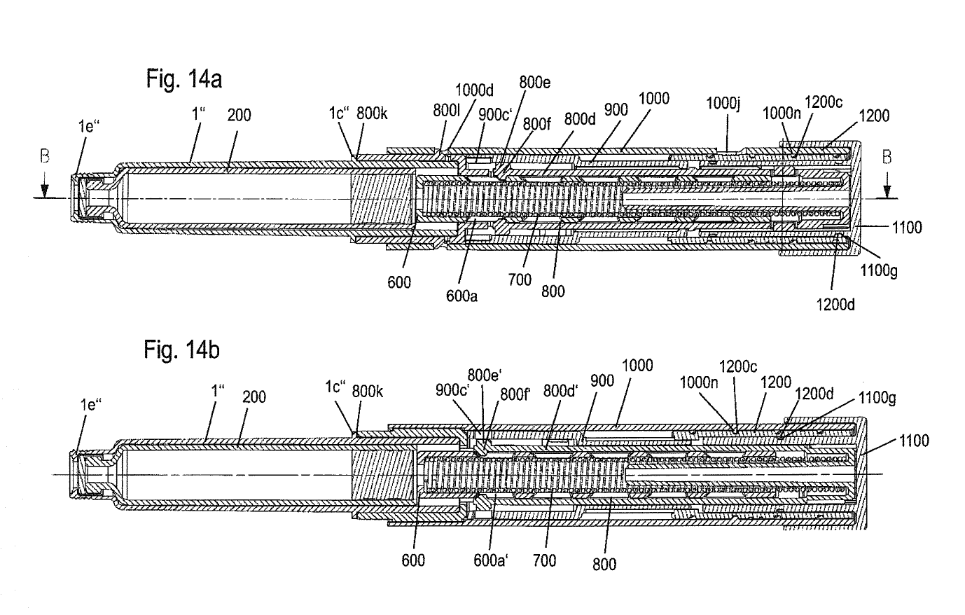

FIG. 15a shows a longitudinal section of the third embodiment of the injection device in a retracted position of the setting button (1100), FIG. 15b showing the injection device according to FIG. 15a, wherein the injection device is rotated about the longitudinal axis B-B by 90°. In order to meter or set the fixed dose, the setting button (1100) is rotated relative to the housing (1000) or the retaining element (800). Because the setting button cam (11000, as can be seen in FIG. 16, is in a guiding engagement or engaged in a slotted guide with the retaining element control cam (800c) of the retaining element (800), the setting button (1100) is screwed by means of the rotation of the setting button (1100) relative to the housing (1000) or the retaining element (800) in the proximal direction until the setting button cam (1100f; visible in FIG. 16, for example) comes into axial alignment with a steep flank of the retaining element control cam (800c) of the control element (800), and the proximal retaining element snap arm (800m) of the retaining element (800) engages with the setting button longitudinal groove or setting button ramp (1100h) of the setting button (1100). Because the pitch of the retaining element control cam (800c) of the retaining element (800) is equally large as the pitch of the threaded connection of the display sleeve internal thread (1200d) and the setting button external thread (1100g), the display sleeve (1200) does not rotate relative to the housing (1000) or the retaining element (800) during the setting movement or the metering movement of the setting button (1100). Because neither the retaining element (800) nor the metering element (900) carries out a movement relative to the housing (1000) during the setting movement or the metering movement of the setting button (1100), the first outward-protruding snap cam (800e) of the first retaining member (800d) of the retaining element (800) remains in stopping contact with the inner circumferential surface of the metering element (900) during the setting movement or the metering movement of the setting button (1200), wherein the first inward-protruding snap cam (800f) of the first retaining member (800d) protrudes into the first recess (600a) of the discharge element (600) and abuts against the distal end face of the connecting web of two recesses arranged offset from one another in the axial direction. The first inward-protruding snap cam (800f) also remains engaged with the first recess (600a) of the discharge element (600), wherein the second inward-protruding snap cam (800f′) of the second retaining member (800d′) elastically relaxes into the second recess (600a′) of the discharge element (600). In addition, the second metering element cutout (900c′) of the metering element (900) still lies above the second outward-protruding snap cam (800e′) of the second retaining member (800d′), the second outward-protruding snap cam (800e′) of the second retaining member (800d′) being disengaged from the second metering element cutout (900c′) of the metering element (900).

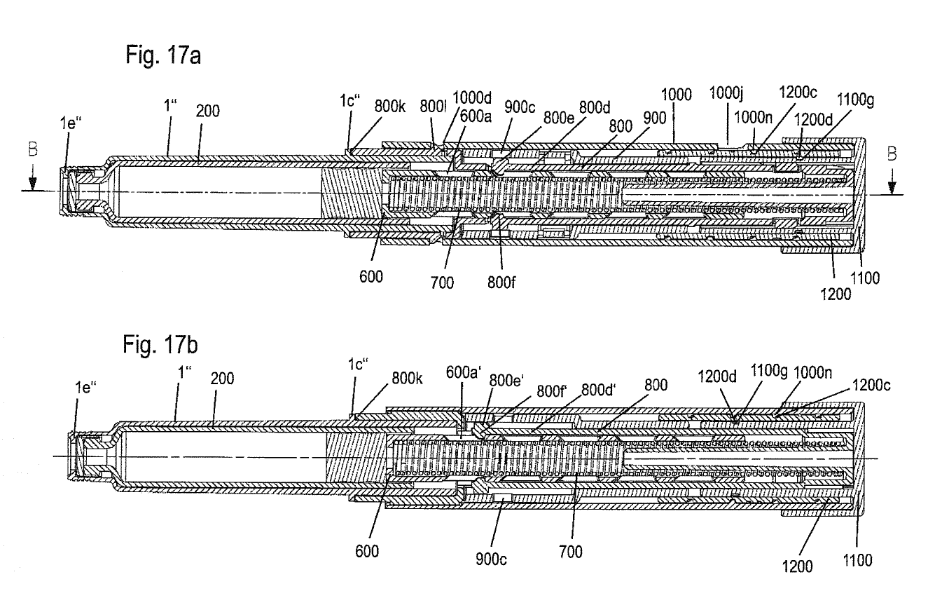

FIG. 17a shows a longitudinal section of the first embodiment of the injection device in an injection-triggered position of the setting button (1100), FIG. 17b showing the injection device according to FIG. 17a, wherein the injection device is rotated about the longitudinal axis B-B by 90°. In order to trigger the fixed dose, the setting button (1100) is moved axially in the distal direction relative to the housing (1000). The display sleeve (1200) rotates due to the axial pressure from the setting button (1100) that acts in the distal direction on the display sleeve (1200) via the two threaded connections between the display sleeve (1200) and the setting button (1100) and between the display sleeve (1200) and the housing (1000). The display sleeve (1200) moves rotationally in the distal direction relative to the housing (1000). The rotatably fixed connection between the display sleeve (1200) and the metering element (900), namely the connection between the proximal metering element groove (900a) and the display sleeve web (1200f), causes the metering element (900) to rotate relative to the housing (1000) or the retaining element (800) during the process of discharging the fixed dose. The metering element (900) rotates relative to the housing (1000) or the retaining element (800) about approximately a 45° angle, so that a first metering element cutout (900c) comes to rest above the first outward-protruding snap cam (800e) of the first retaining member (800d) of the retaining element (800), and another first metering element cutout (900c) comes to rest axially offset from the second outward-protruding snap cam (800e′) of the first retaining member (800d′) of the retaining element (800). The circumferential surface of the metering element (900) lies above the second outward-protruding snap cam (800e′) of the second retaining member (800d′) of the retaining element (800), the metering element above the second outward-protruding snap cam (800e′) preventing the second inward-protruding snap cam (800f′) of the second retaining member (800d′) from disengaging from the second cutout (600a′) of the discharge element (600).

FIG. 18 shows a detailed view of the proximal end of the injection device of the third embodiment in a completely discharged position of the setting button (1100), wherein a part of the housing (1000) and a part of the setting button (1100) are cut away so that the interior of the injection device is visible. After the final dose has been discharged from the injection device, the housing internal thread start (1000p) of the housing internal thread (1000n) is in the vicinity of the display sleeve external thread end (1200e) of the display sleeve external thread (1200c). During the setting movement or the retracting movement of the setting button (1100) for an additional fixed dose, the display sleeve (1200) does not rotate relative to the housing (1000) or the retaining element (900) while the setting button (1100) is being screwed in the proximal direction relative to the housing (1000) or the retaining element (900). Thus the display sleeve (1200) does not move axially in the distal direction relative to the housing (1000) or the retaining element (900). During the triggering movement of the setting button (1100) to discharge an additional fixed dose, the housing internal thread start (1000p) of the housing (1000) strikes against the display sleeve thread end (1200e), so that the display sleeve (1200) cannot carry out a rotation relative to the housing (1000) or the retaining element (900), and consequently, the metering element (900) is likewise not rotated relative to the housing (1000) or the retaining element (900). Consequently, no further fixed dose can be discharged from the injection device.

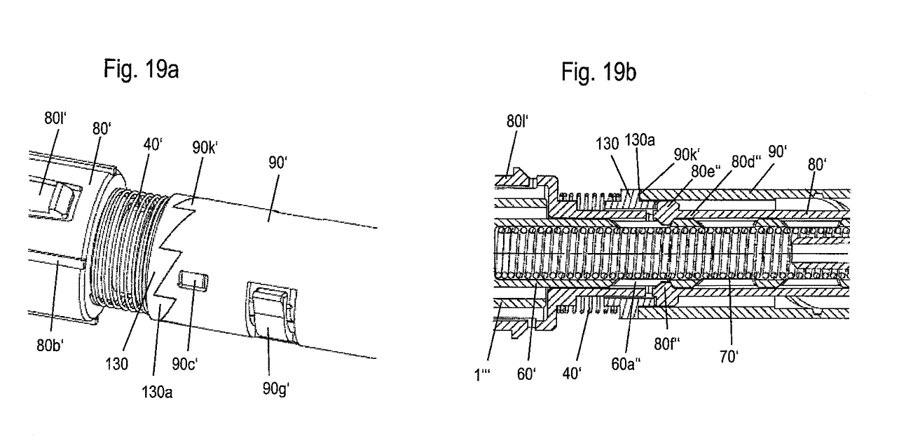

FIGS. 19a-21a show a mechanism for indicating the end of injection in the various stages, wherein the mechanism of this display can be installed in the injection device according to the third embodiment, for example.

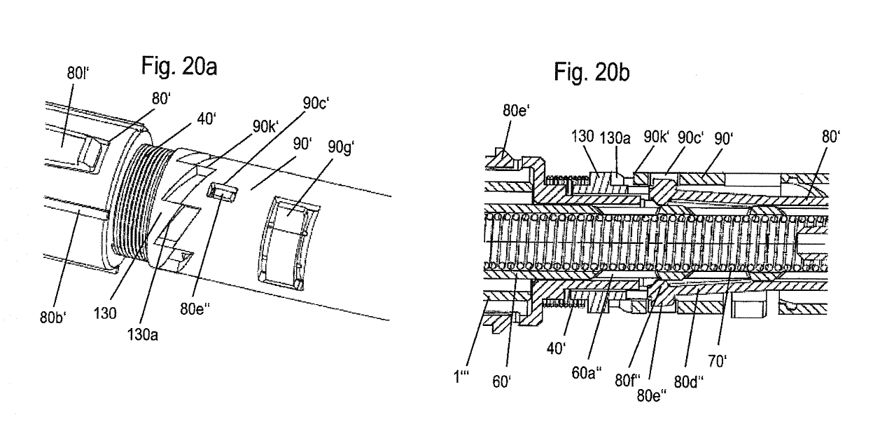

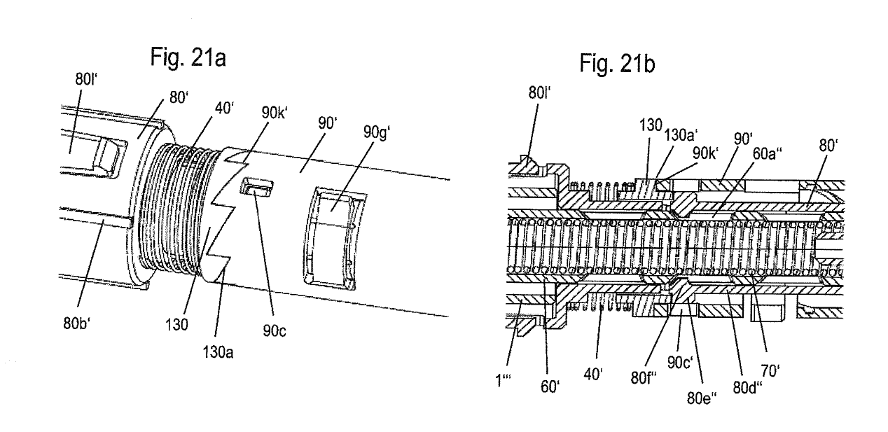

The injection device can comprise a spring (40′), which is supported distally on a retaining element (80′) fixed in relation to the housing and proximally on a click element (130). The retaining element (80′) is connected axially fixedly and rotationally fixedly to a housing via a distal retaining element snap arm (80l′) and via a retaining element web (80b′). The retaining element (80′) is additionally axially fixedly and rotationally fixedly connected to a carpule holder (1′″). The click element (130) is connected rotationally fixedly to the retaining element (80′). The click element (130) is mounted axially movably on the retaining element (80′). The click element (130) is prestressed by a spring force against a distal end face of the metering element (90′). A sawtooth-like metering element control cam (90k′) is provided on a distal end of the metering element (90′), and a sawtooth-like click element control cam (130a) is provided on the proximal end of the click element (130). The sawtooth-like metering element control cam (90k′) of the metering element (90′) and the sawtooth-like click element control cam (130a) of the click element (130) are formed complementarily to one another and can interlock. In the initial position of the setting button, the sawtooth-like metering element control cam (90k′) engages with the click element control cam (130a), as shown in FIGS. 19a and 19b. The circumferential surface of the metering element lies above the first outward-protruding snap cam (80e″) of the first retaining member (80d″) of the first retaining element (80″) in such a manner that the first inward-protruding snap cam (80f″) of the first retaining member (80d″) of the retaining element (80″) does not disengage from a first recess (60a″) of a discharge element (60′). The discharge element (60′), which is prestressed in the distal direction by a prestressed spring (70′), is prevented from moving relative to the housing or the retaining element (80′) by the stopping contact between a proximal end face of the first inward-protruding snap cam (80f″) of the first retaining member (80d″) of the retaining element (80′) and a distal end face of a connecting web between two recesses (60a″) of the discharge element (60′) that are arranged offset from one another in the axial direction. During the process of discharging the fixed dose from the injection device, as shown in FIG. 20a and in FIG. 20b, the metering element (90′) rotates relative to the retaining element (80′). The metering element (90′) can be rotated in only one direction due to the connection between a metering element snap arm (90g′) and a housing ramp of the housing. Due to the sawtooth-like engagement between the click element (130) and the metering element (90′) and due to the rotationally fixed connection between the click element (130) and the retaining element (80′), the click element (130) can be moved axially in the distal direction relative to the housing or the retaining element (90′), so that the spring (40″) is thus further pretensioned. By means of the relative rotation of the metering element (90′), the first metering element cutout (90c′) moves over the first outward-protruding snap cam (80e″) of the first retaining member (80d″) of the retaining element (80), wherein the first outward-protruding snap cam (80e″) is deflected elastically into the first metering element cutout (90c′), and the first inward-protruding snap cam (80f″) of the first retaining member (80d″) of the retaining element (80) disengages from the first cutout (60a′) of the discharge element (60′). This deflection of the first outward-protruding snap cam (80e″) into the first metering element cutout (90c′) has the effect that the click element can be held in the distal position. The connecting web of two first recesses (60a′) that are arranged axially offset from one another slides along the first inward-protruding snap cam (80f″) of the first retaining member (80d″) of the retaining element (80) until the first inward-protruding snap cam (80f″) deflects elastically into the more proximal first recess (60a′) of the discharge element (60), as shown in FIGS. 21a and 21b. The click element (130) is no longer held in the distal position, and due to the force of the prestressed spring (40′) is able to strike with the click element control cam (130a) against the metering element control cam (90k′) of the metering element (90′). This stop can trigger an acoustic signal, in particular a click sound. This acoustic signal indicates the end of injection of the fixed dose. The discharge element (60′) is prevented from further axial movement relative to the housing or the retaining element (80′) due to a stopping contact between a second inward-protruding snap cam of a second retaining member (not visible) of the retaining element (80) and a distal end face of a connecting web of two second recesses (not visible) of the discharge element (60′) that are arranged offset from one another in the axial direction.

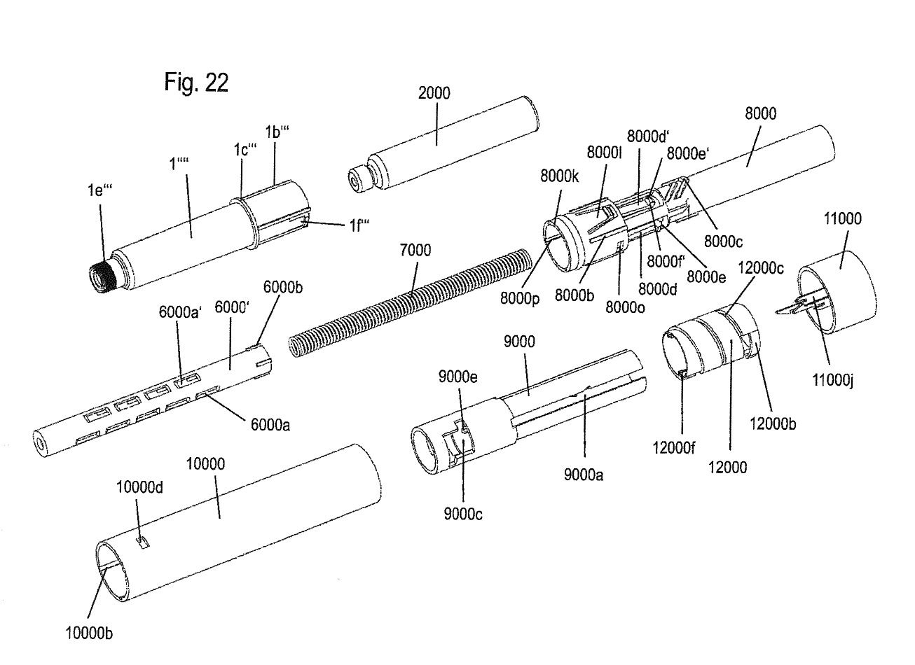

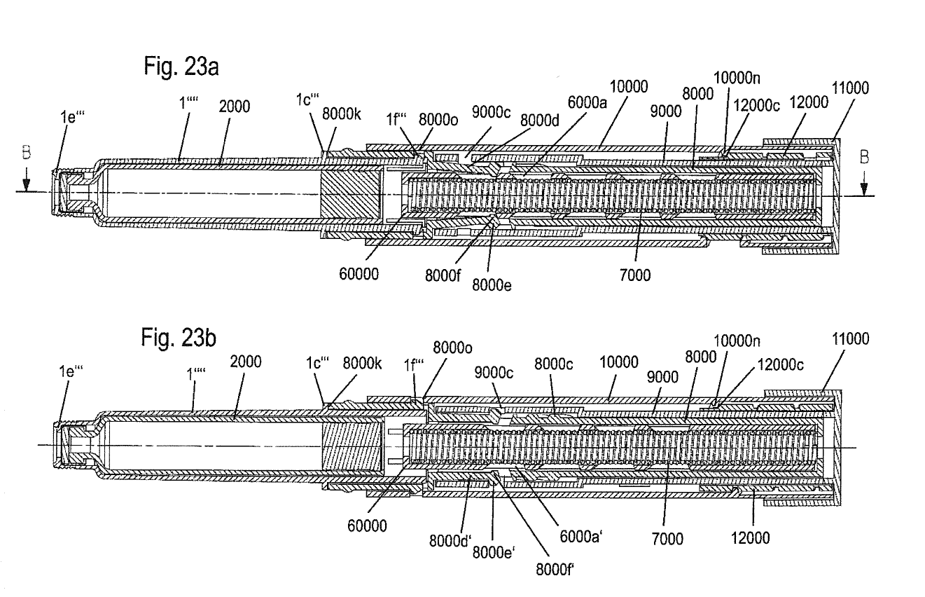

FIG. 22 shows an exploded view of a fourth embodiment of an injection device according to the invention. A carpule holder (1″″) has received a carpule (2000), wherein the carpule holder (1″″) is axially fixedly and rotationally fixedly connected via a carpule holder snap arm (1f′″) and a carpule holder web (1b′″) to a retaining element cutout (8000o) provided on the inner circumferential surface of a retaining element (8000) and a distal retaining element groove (8000p) provided on the inner circumferential surface of the retaining element (8000). A carpule holder stop (1c′″) can strike axially against a distal retaining element edge (8000k). A needle-connecting element (1e′″) for releasably mounting an injection needle is provided on the distal end of the carpule holder (1″″). The injection device further comprises a metering element (9000), the metering element (9000) being provided radially between a housing (10000) and the retaining element (8000). The retaining element (8000) is rotationally fixedly connected, with the aid of a retaining element web (8000b) on the retaining element (8000), to the housing (10000) via a distal housing groove (10000b). The axially fixed connection between the retaining element (8000) and the housing (10000) is accomplished via a connection between a distal retaining element snap arm (8000l) and a proximal housing cutout (10000d). The injection device further comprises a setting button (11000), which is axially fixedly and rotationally fixedly secured via a setting button securing element (11000j) of the setting button (11000) with a metering element securing element (not visible) arranged inside the metering element (9000). The injection device further comprises a display sleeve (12000), which has a threaded connection to the housing (10000). For this purpose, the display sleeve (12000) comprises a display sleeve external thread (12000c), which is engaged with a housing internal thread (10000n; visible in FIG. 23a or 23b, for example) of the housing (10000). The display sleeve (1200) additionally has, on the inner circumferential surface thereof, a display sleeve web (12000f), which is engaged with a proximal metering element groove (9000a) of the metering element (9000) for a rotationally fixed connection. A rotation of the metering element (9000) thus causes a rotation of the display sleeve (12000). The display sleeve (12000) additionally comprises a display sleeve snap arm (12000b), which can interact with a proximal housing groove or proximal housing ramp (not visible) in such a manner that the display sleeve (12000) can be rotated in only one rotational direction. This cooperation can produce an acoustic sound, in particular a click sound, in order to indicate the setting or metering of the fixed dose. For visually displaying the setting or metering of the fixed dose, the display sleeve (12000) can additionally or alternatively have a display digit and/or a marking and/or an intermediate space (not shown), which can be visible through a housing cutout (not shown) of the housing (10000). The metering element (9000) has a metering element cam (9000e), which is in a guiding engagement or is engaged via a slotted guide with a retaining element control cam (8000c) of the retaining element (8000). The retaining element control cam (8000c) of the retaining element (8000) is formed such that the metering element (9000) is axially movable in the proximal direction relative to the housing (10000) by a relative rotation of the metering element (9000). A metering element cutout (9000c), which is suitable for receiving a first outward-protruding snap cam (8000e) and/or a second outward-protruding snap cam (8000e′) of a first retaining member (8000d) and a second retaining member (8000d′) of the retaining element (8000), is provided in the metering element (9000). The first retaining member (8000d) and the second retaining member (8000d′) can be constructed as a snap arm or a snap tongue. The first retaining member (8000d) and the second retaining member (8000d′) are arranged relative to one another at approximately a 90° angle in the circumferential direction of the retaining element (8000). A first inward-protruding snap cam (8000f; visible in FIG. 23a, for example) is arranged on the first retaining member (8000d) approximately diametrically opposite the first outward-protruding snap cam (8000e), while a second inward-protruding snap cam (8000f′; visible in FIG. 23b, for example) is provided on the second retaining member (8000d′) approximately diametrically opposite the second outward-protruding snap cam (8000e′). In addition, two first retaining members (8000d) and two second retaining members (8000d′) are provided on the retaining element (8000), wherein the two first retaining members (8000d) are arranged at approximately a 180° angle relative to one another in the circumferential direction of the retaining element (8000), and the two second retaining members (8000d′) are arranged at approximately a 180° angle relative to one another in the circumferential direction of the retaining element (8000). Both first retaining members (8000d) and both second retaining members (8000d′) each comprise an outward-protruding snap cam (8000e; 8000e′) and an inward-protruding snap cam (8000f; 8000f′). The first outward-protruding snap cam (8000e) and the second outward-protruding snap cam (8000e′) and the first inward-protruding snap cam (8000f) and the second inward-protruding snap cam (8000f′) can be constructed as snap hooks. The injection device further comprises a discharge element (6000), which has received a prestressed spring (7000). A first recess (6000a) and a second recess (6000a′) are provided in the discharge element (6000). The first recess (6000a) and the second recess (6000a′) of the discharge element (6000) are arranged offset from one another in the circumferential direction and the axial direction, wherein a plurality of first recesses (6000a) and a plurality of second recesses (6000a′) are provided, respectively arranged offset from one another in the axial direction. The first recess (6000a) and the second recess (6000a′) are arranged relative to one another at approximately a 90° angle in the circumferential direction of the discharge element (6000). The first recess (6000a) is suitable for receiving the first inward-protruding snap cam (8000f; visible in FIG. 23a, for example) and the second recess (6000a′) is suitable for the second inward-protruding snap cam (8000f′; visible in FIG. 23b, for example). The first recess (6000a) and the second recess (6000a′) of the discharge element (6000) overlap one another in the axial direction. In addition, the discharge element (6000) comprises two first recesses (6000a), which are arranged offset from one another by an angle of approximately 180° in the circumferential direction of the discharge element (6000), wherein the discharge element (6000) provides a plurality of first recesses (6000a) arranged offset axially from one another. Furthermore, the discharge element (6000) comprises two second recesses (6000a′), which are arranged offset from one another by an angle of approximately 180° in the circumferential direction of the discharge element (6000), wherein the discharge element (6000) provides a plurality of second recesses (6000a′) arranged offset axially from one another. The discharge element (6000) further comprises a discharge element web (6000b), which forms a rotationally fixed connection with a retaining element longitudinal groove (not visible) provided on the inner circumferential surface.

FIG. 23a shows a longitudinal section of the fourth embodiment of the injection device in an initial position of a metering element (9000), FIG. 23b showing the injection device according to FIG. 23a, wherein the injection device is rotated about the longitudinal axis B-B by 90°. The first retaining member (8000d) is tensioned elastically with the first inward-protruding snap cam (8000f) by the circumferential surface of the metering element (9000) and the first outward-protruding snap cam (8000e) of the first retaining member (8000d) of the retaining element (8000) into the first recess (6000a) of the discharge element (6000), so that the first inward-protruding snap cam (8000f) is engaged with the first recess (6000a) of the discharge element (6000). A proximal end face of the first inward-protruding snap cam (6000f) of the first retaining member (8000d) of the retaining element (8000) is in stopping contact with a connecting web of two recesses (6000a) of the discharge element (6000) that are arranged axially offset from one another, so that the discharge element (6000), prestressed with the spring (7000), is prevented from moving axially relative to the housing (10000) or the retaining element (8000). The second retaining member (8000d′) is elastically relaxed, so that the second outward-protruding snap cam (8000e′) protrudes into the metering element cutout (9000c) of the metering element (9000), and the second inward-protruding snap cam (8000f′) is disengaged from the second cutout (6000a′) of the discharge element (6000). The metering element cam (9000e) of the metering element (9000) is in guiding engagement or engaged via the slotted guide with the retaining element control cam (8000c) of the retaining element (8000) such that the metering element cam (9000e) and the retaining element control cam (8000c) may be configured as a guide element and a counter-guide element, respectively.

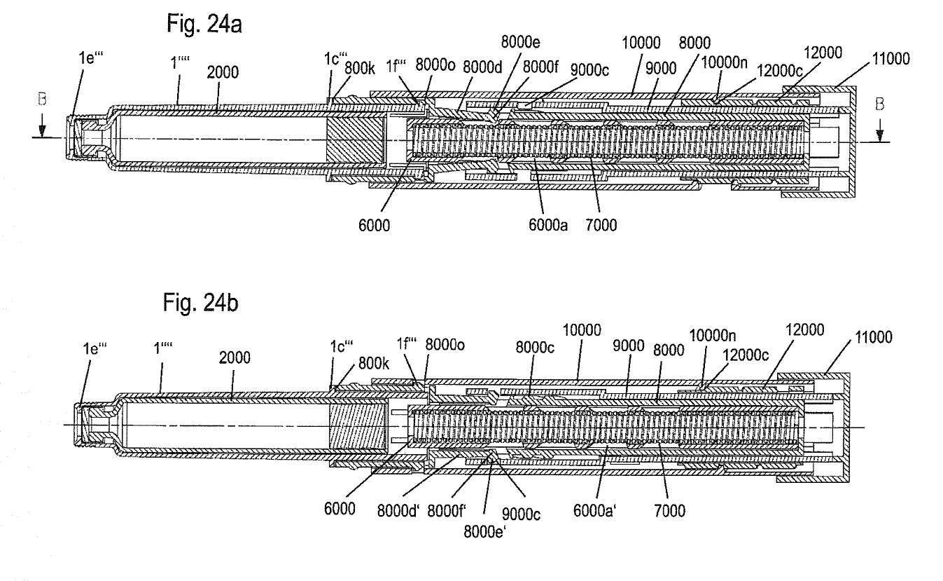

FIG. 24a shows a longitudinal section of the fourth embodiment of the injection device in a retracted position of the metering element (9000), FIG. 24b showing the injection device according to FIG. 24a, wherein the injection device is rotated about the longitudinal axis B-B by 90°. To make the injection device ready for injection, the user rotates the setting button (11000) relative to the housing (10000). Due to the rotationally fixed connection between the setting button (11000) and the metering element (9000), the metering element (9000) and the setting button (11000) are moved in the proximal direction relative to the housing (10000) or the retaining element (8000) via the guiding engagement or the slotted guide engagement between the metering element cam (9000e) of the metering element (9000) and the retaining element control cam (8000c) of the retaining element (8000), which is fixed relative to the housing. Since the retaining element control cam (8000c) of the retaining element (8000) is formed helically, the setting button (11000) and the metering element (9000) are screwed in the proximal direction out of the housing (10000). Due to the rotationally fixed connection between the metering element (9000) and the display sleeve (12000) and to the threaded connection between the display sleeve (12000) and the housing (10000), the display sleeve (12000) is screwed in the distal direction relative to the housing (1000). In the retracted position of the metering element (9000), the first inward-protruding snap cam (8000e) of the first retaining member (8000d) is still kept engaged by the metering element (9000) with the first cutout (6000a) of the discharge element (6000) via the first inwardly-protruding snap cam (8000f) of the first retaining member (8000d), wherein the metering element cutout (9000c) of the metering element (9000) comes into axial alignment with, and is offset in the proximal direction from, the first outward-protruding snap cam (8000e) of the first retaining member (8000d), due to the relative rotational movement of the metering element (9000). In addition, the second outward-protruding snap cam (8000e′) of the second retaining member (8000d′) remains in the metering element cutout (9000c) of the metering element (9000), so that the second inward-protruding snap cam (8000f′) of the second retaining element (8000d′) is disengaged from the second cutout (6000a′) of the discharge element (6000).

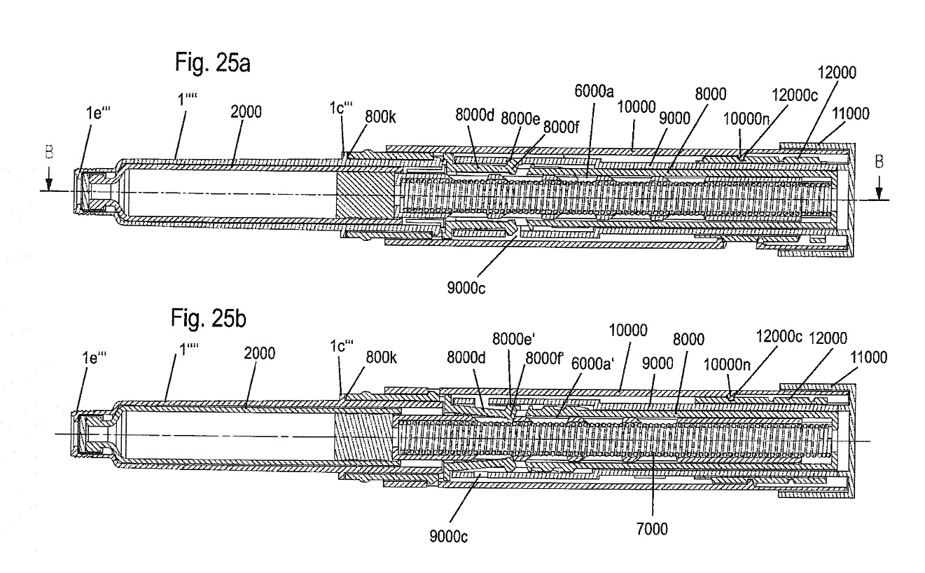

FIG. 25a shows a longitudinal section of the fourth embodiment of the injection device in an injection-triggered position of the metering element (9000), FIG. 25b showing the injection device according to FIG. 25a, wherein the injection device is rotated about the longitudinal axis B-B by 90°. To trigger the injection of the fixed dose, the user actuates the setting button (11000) by moving the setting button (11000) axially in the distal direction relative to the housing (10000) or the retaining element (8000). Due to this axial movement, the metering element cutout (9000c) of the metering element (9000) moves above the first outward-protruding snap cam (8000e) of the retaining element (8000) in such a manner that the first outward-protruding snap cam (8000e) of the retaining element (8000) is elastically deflected into the metering element cutout (9000c) of the metering element (9000), wherein the first retaining member (8000d) elastically relaxes. The discharge element (6000) moves axially in the distal direction relative to the housing (10000) during the process for discharging the fixed dose. During the discharging process, the circumferential surface of the metering element (9000) moves above the second outward-protruding snap cam (8000e′) of the second retaining member (8000d′) of the retaining element (8000), wherein the second retaining element (8000d′) with the second inward-protruding snap cam (8000f′) is elastically deflected or relaxes elastically into the second recess (6000a′) of the discharge element (6000). The discharge element (6000) moves in the distal direction until a proximal end face of the second inward-protruding snap cam (8000f′) comes into stopping contact against a distal end face of a connecting web of two second recesses (6000a′) of the discharge element (6000) that are arranged axially offset from one another. During the discharge movement, the display sleeve (12000) does not rotate relative to the housing (10000) or the retaining element (8000). If setting or metering another fixed dose is attempted when the final available fixed dose has been discharged from the injection device, a distal end face of the display sleeve (12000) strikes against a stop positioned on the inner circumferential surface of the housing (10000), so that a further fixed dose cannot be set or metered.

While the present disclosure has been described with reference to various embodiments, it will be understood that these embodiments are illustrative and that the scope of the disclosure is not limited to them, and variations, modifications, additions, and improvements are possible. More generally, embodiments in accordance with the present disclosure have been described in the context or particular embodiments. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. An injection device for metering and discharging a fixed dose of a liquid product, comprising:
a metering element or a setting button for setting and discharging a fixed dose comprising a guide element;
a retaining element comprising a counter-guide element, a first retaining member and a second retaining member; and
a discharge element for discharging a fixed dose comprising a first recess for receiving at least a portion of the first retaining member, and a second recess for receiving at least a portion of the second retaining member,
wherein the first recess of the discharge element cooperates with at least the portion of the first retaining member of the retaining element, and the second recess of the discharge element cooperates with at least the portion of the second retaining member of the retaining element such that:
in an initial position of the metering element or of the setting button, in which the metering element or the setting button is in a distal position, at least the portion of the first retaining member of the retaining element is engaged with the first recess of the discharge element,
in a retracted position of the metering element or of the setting button, in which the metering element or the setting button is in a proximal position, at least the portion of the first retaining member of the retaining element is engaged with the first recess of the discharge element, and
in a discharged position of the metering element or the setting button, in which the metering element or the setting button is in a distal position, at least the portion of the second retaining member of the retaining element is engaged with the second recess of the discharge element, and
wherein in the initial position, in the retracted position and in the discharged position, the guide element of the metering element or of the setting button is in a guiding engagement or in a slotted guide engagement with the counter-guide element of the retaining element.

2. The injection device according to claim 1, wherein the injection device comprises each of the metering element and the setting button, and wherein the metering element and the setting button are indirectly or directly coupled to one another.

3. The injection device according to claim 1, wherein the injection device comprises at least the metering element, and wherein the metering element further comprises a cutout formed such that, in the discharged position, the first retaining member is engaged with the cutout.

4. The injection device according to claim 3, wherein in the initial position, the metering element is in sliding contact with the first retaining member.

5. The injection device according to claim 4, wherein in the retracted position, the metering element is in sliding contact with the first retaining member.

6. The injection device according to claim 3, wherein in the retracted position, the metering element is in sliding contact with the first retaining member.

7. The injection device according to claim 1, wherein the metering element or the setting button is rotatable relative to the retaining element.

8. The injection device according to claim 1, wherein the device further comprises a prestressed spring, which acts on the discharge element in the initial position of the metering element or of the setting button.

9. The injection device according to claim 1, wherein the discharge element comprises a sleeve-like shape.

10. The injection device according to claim 1, wherein the first recess and the second recess of the discharge element are arranged relative to the first retaining member and the second retaining member such that the discharge element and the first and second retaining members cooperate to enable a fixed dose to be discharged from the device.

11. The injection device according to claim 10, wherein the first recess and the second recess of the discharge element are arranged offset from one another circumferentially and axially.

12. The injection device according to claim 11, wherein the retaining element comprises a sleeve-like shape.

13. The injection device according to claim 12, wherein the first retaining member and the second retaining member of the retaining element are arranged offset from one another circumferentially and at a same axial height or at different axial heights.

14. The injection device according to claim 13, wherein the first retaining member and the second retaining member of the retaining element are elastically arranged and configured to engage with the respective first and second recesses of the discharge element.

15. The injection device according to claim 14, wherein the first retaining member and the second retaining member of the retaining element comprise a snap arm or a snap tongue and comprise at least one snap hook, snap cam or protrusion.

16. The injection device according to claim 1, wherein the guide element of the metering element or of the setting button is formed as one of a guide cam or as a slotted guide, and wherein the counter-guide element of the retaining element is formed as the other of the guide cam or the slotted guide.
